(19) United States
(12) Patent Application Publication
(10) Pub. No.: US 2015/0018301 A1
(43) Pub. Date: Jan. 15, 2015

(54) LRRK-2-Mediated Neuronal Toxicity

(71) Applicants: Byoung Dae Lee, Cockeysville, MD (US); Ted M. Dawson, Baltimore, MD (US); Valina L. Dawson, Baltimore, MD (US); Andrew B. West, Birmingham, AL (US); Howard Federoff, Bethesda, MD (US)

(72) Inventors: Byoung Dae Lee, Cockeysville, MD (US); Ted M. Dawson, Baltimore, MD (US); Valina L. Dawson, Baltimore, MD (US); Andrew B. West, Birmingham, AL (US); Howard Federoff, Bethesda, MD (US)

(73) Assignee: THE JOHNS HOPKINS UNIVERSITY, Baltimore, MD (US)

(21) Appl. No.: 13/508,180

(22) PCT Filed: Nov. 8, 2010
(86) PCT No.: PCT/US10/55857

§ 371 Date: Jul. 12, 2013

Related U.S. Application Data
(60) Provisional application No. 61/258,725, filed on Nov. 6, 2009.

(51) Int. Cl. A61K 31/553; A61K 31/404; A61K 31/7064; A61K 31/122; A61K 31/416

(52) U.S. Cl. CPC A61K 31/553; A61K 31/122; A61K 31/416; A61K 31/7064; A61K 31/404
USPC 514/49; 435/184; 514/410; 514/680; 514/406; 514/414; 514/418

(57) ABSTRACT

Leucine-rich repeat kinase-2 (LRRK2) mutations are a common cause of Parkinson's disease. Inhibitors of LRRK2 kinase that are protective in in vitro and in vivo models of LRRK2-induced neurodegeneration were identified. The presently disclosed subject matter establishes that LRRK2-induced degeneration of neurons in vivo is kinase dependent and that LRRK2 kinase inhibition provides a potential new neuroprotective paradigm for treating Parkinson's disease.

### CROSS-REFERENCE TO RELATED APPLICATIONS

This application claims the benefit of U.S. Provisional Application No. 61/258,725, filed Nov. 6, 2009; which is incorporated herein by reference in its entirety.

### FEDERALLY SPONSORED RESEARCH OR DEVELOPMENT

This invention was made in part with United States government support under NS 38377 awarded by the National Institutes of Health (NIH)/National Institute of Neurological Disorders and Stroke (NINDS). The government has certain rights in the invention.

### BACKGROUND

Parkinson's disease is a very common neurodegenerative disorder with no proven neuroprotective or neurorestorative therapies. Recent advances in identifying genetic causes of Parkinson's disease have provided new opportunities for discovery of therapeutic targets and agents to potentially prevent the degenerative process of Parkinson's disease. Individuals with leucine-rich repeat kinase-2 (LRRK2) mutations are clinically and neurochemically indistinguishable from those with idiopathic Parkinson's disease. Gasser, T. Expert Rev. Mol. Med. 11, e22 (2009).

Disease-segregating mutations in LRRK2 lead to neurotoxicity in vitro, Greggio, E. et al. Neurobiol. Dis. 23, 329-341 (2006); Smith, W. W. et al. Nat. Neurosci. 9, 1231-1233 (2006); West, A. B. et al. Hum. Mol. Genet. 16, 223-232 (2007), and loss of dopamine neurons in subject afflicted with Parkinson's disease. Whaley, N. R., Uitti, R. J., Dickson, D. W., Farrer, M. J. &. Wszolek, Z. K. J. Neural Transm. Suppl. 70, 221-229 (2006).

LRRK2 toxicity in vitro is linked to kinase activity and GTP binding, as mutations in LRRK2 that interfere with kinase activity and GTP binding inhibit toxicity. Whether LRRK2 toxicity requires kinase activity in vivo and whether pharmacologic inhibition of this activity would protect against LRRK2 toxicity is not known.

### SUMMARY

In some aspects, the presently disclosed subject matter provides a method for inhibiting a leucine-rich repeat kinase-2 (LRRK2) kinase, the method comprising contacting an LRRK2 kinase with a compound of formulae (I-VII):

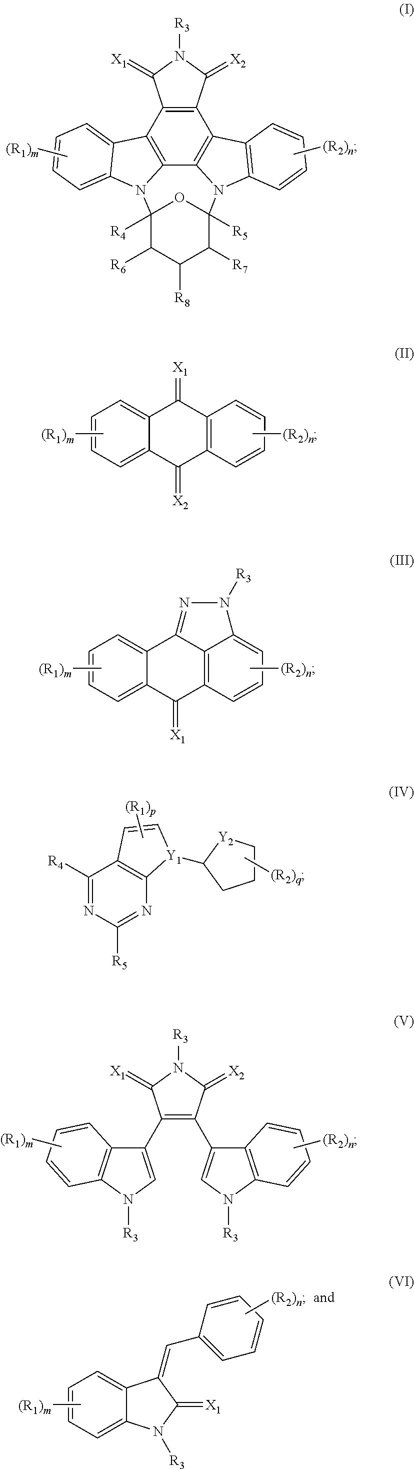

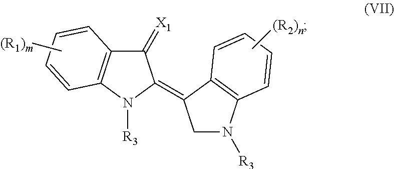

wherein p is an integer selected from the group consisting of 0, 1, and 2; q is an integer selected from the group consisting of 0, 1, 2, and 3; m and n are each an integer independently selected from the group consisting of 0, 1, 2, 3, and 4; each (=X₁) and (=X₂) can be present or absent and, when present, each X₁ and X₂ is independently selected from the group consisting of O, S, CR₉R₁₀, and NR₁₁, wherein R₉, R₁₀, and R₁₁ are each independently selected from the group consisting of H, substituted or unsubstituted alkyl, substituted or substituted heteroalkyl, substituted or substituted alkenyl, alkynyl, substituted or unsubstituted cycloalkyl, substituted or unsubstituted cycloheteroalkyl, substituted or unsubstituted aryl, substituted or unsubstituted heteroaryl, aralkyl, hydroxyl, —COR₁₂, —COOR₁₃, —OR₁₄, wherein R₁₂, R₁₃, and R₁₄ are each independently selected from the group consisting of H, substituted or unsubstituted alkyl, substituted or substituted heteroalkyl, substituted or substituted alkenyl, alkynyl, substituted or unsubstituted cycloalkyl, substituted or unsubstituted cycloheteroalkyl, substituted or unsubstituted aryl, substituted or unsubstituted heteroaryl; Y₁ is selected from the group consisting of N, and CR₉, wherein R₉ is as defined above; Y₂ is selected from the group consisting of O, S, CR₉R₁₀, and NR₁₁, wherein R₉, R₁₀, and R₁₁ are as defined above; each R₁, R₂, R₄, R₅, R₆, R₇, and R₈ is independently selected from the group consisting of substituted or unsubstituted alkyl, substituted or substituted heteroalkyl, substituted or substituted alkenyl, alkynyl, substituted or unsubstituted cycloalkyl, substituted or unsubstituted cycloheteroalkyl, substituted or unsubstituted aryl, substituted or unsubstituted heteroaryl, arylalkyl, arylalkynyl, alkoxyl, acyloxyl, aryloxyl, arylalkyloxyl, cycloalkylalkyloxyl, cycloalkyloxyl, alkoxyalkyl, alkoxyalkoxyl, aminoalkoxyl, mono- or di-alkylaminoalkoxyl, alkoxycarbonyl, carboxyl, halo, amino, alkylamino, acylamino, arylamino, sulfonyl, arylmercapto, alkylmercapto, hydroxyl, hydroxyalkyl, hydroxycycloalkyl, alkoxycycloalkyl, aminoalkyl, alkylaminoalkyl, cyano, nitro, CF₃, —COR₁₂, —COOR₁₃, and —OR₁₄, wherein R₁₂, R₁₃, and R₁₄ are as defined above; R₃ is selected from the group consisting of H, substituted or unsubstituted alkyl, substituted or substituted heteroalkyl, substituted or substituted alkenyl, alkynyl, substituted or unsubstituted cycloalkyl, substituted or unsubstituted cycloheteroalkyl, substituted or unsubstituted aryl, substituted or unsubstituted heteroaryl, aralkyl, hydroxyl, —COR₁₂, —COOR₁₃, —OR₁₄, wherein R₁₂, R₁₃, and R₁₄ are as defined above; and stereoisomers, prodrugs, and pharmaceutically acceptable salts thereof.

In other aspects, the presently disclosed subject matter provides a method for treating a disorder or a condition that can be treated by inhibiting LRRK2 activity in a subject in need of treatment thereof, the method comprising administering to the subject a therapeutically effective amount of a compound of formulae (I-VII). In certain aspects, the disorder or a condition that can be treated by inhibiting LRRK2 activity comprises a neurodegenerative disease. In particular aspects, the disorder or condition that can be treated by inhibiting LRRK2 activity is Parkinson's disease. In other aspects, the disorder or a condition that can be treated by inhibiting LRRK2 activity comprises an autoimmune disease. In particular aspects, the autoimmune disease comprises Crohn's disease.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIGS. 1a-1i are (a) LRRK2 autophosphorylation (% of control) in the presence or absence of kinase inhibitors (Table 1). ***P<0.001 by analysis of variance (ANOVA) compared to the other groups. Neuman-Keuls post hoc test. Degrees of freedom=34 (total) and F=18.4144; (b) Representative phosphoimage of WT LRRK2 and LRRK2 G2019S autophosphorylation in the presence or absence of LRRK2 kinase inhibitors. LRRK2 kinase-dead (D1994A) and KN-93 are negative controls. IB, immunoblot; (c, d) Dose-response curves of WT LRRK2 and G2019S LRRK2 autophosphorylation after treatment with LRRK2 kinase inhibitors; (e-g) Raf kinase inhibitors dose-response curves on WT LRRK2 (e), G2019S LRRK2 (f) and LRRK1 (g) autophosphorylation; (h) G2019S LRRK2 autophosphorylation and 4E-BP1 phosphorylation in the presence or absence of LRRK2 kinase inhibitors. The G2019S LRRK2 kinase-dead mutant (G2019S-D1994A), ZM336372 and indirubin are negative controls; (i) Quantification of G2019S LRRK2 autophosphorylation and 4E-BP1 phosphorylation in the presence or absence of LRRK2 kinase inhibitors, ***P<0.001, by ANOVA, Neuman-Keuls post hoc test. Degrees of freedom for LRRK2=17 (total) and F=22.401. Degrees of freedom for 4E-BP1=17 (total) and F=22.453. All data represent the mean±S.E.M. from three independent experiments;

FIGS. 2a-2f show screening of Biomol kinase and phosphatase inhibitor library to identify potential LRRK2 inhibitors;

FIG. 2a shows screening of Biomol kinase and phosphatase inhibitor library to identify the potential LRRK2 kinase inhibitors. LRRK2-mediated myelin basic protein (MBP) phosphorylation (% of control). Quantitation of MBP phosphorylation normalized by MBP phosphorylation in the absence of compound. Data represent the mean±S.E.M from three independent experiments. LRRK2 and MBP were incubated with [γ-32P] ATP in the absence or presence of compounds (16 μM) at 30° C. for 15 min. Incorporation of 32P in MBP was detected by use of a Phosphoimager and images were analyzed by ImageQuant 6.0 software. The list of compounds is supplied in Table 1. ***p<0.001 by ANOVA compared to control activity. Neuman-Keuls post hoc test. Degree of freedom=27 (total) and F=28.3085;

FIG. 2b shows representative phosphoimage of LRRK2 WT and LRRK2 G2019S-mediated MBP phosphorylation in the presence and absence of kinase inhibitors. LRRK2 kinase dead (D1994A) is included as a positive control and the kinase inhibitor KN-93, which has no effect on LRRK2 kinase activity, is included as a negative control. Coomassie brilliant blue staining (CBB) is shown as a loading control for MBP. Autoradiographs and blot are representative of three independent experiments;

FIG. 2c shows dose-response curves of the inhibition of LRRK2 WT mediated MBP phosphorylation of the eight LRRK2 kinase inhibitors identified above in FIG. 2a and FIG. 2b. Data represent the mean±S.E.M. from three independent experiments;

FIG. 2d shows dose-response curves of the inhibition of LRRK2 G2019S-mediated MBP phosphorylation of the eight LRRK2 kinase inhibitors identified above in FIG. 2a and FIG. 2b. Data represent the mean±S.E.M. from three independent experiments;

FIG. 2e shows dose-response curves of the effect of Raf kinase inhibitors on LRRK2 WT mediated phosphorylation of MBP. Data represent the mean±S.E.M. from three independent experiments;

FIG. 2f shows dose-response curves of the effect of Raf kinase inhibitors on LRRK2 G2019S mediated phosphorylation of MBP. Data represent the mean±S.E.M. from three independent experiments;

FIGS. 3a-3e show differential effects of kinase inhibitors on LRRK2 versus LRRK1-mediated phosphorylation of MBP;

FIG. 3a shows representative phosphoimage of LRRK1 WT autophosphorylation in the presence and absence of kinase inhibitors. The kinase inhibitor KN-93, which has no effect on LRRK1 kinase activity, is included as a negative control. Immunoblot (IB) against GST is shown as a loading control. Autoradiography and blot are representative of three independent experiments;

FIG. 3b shows percent inhibition of LRRK1 and LRRK2 kinase activity by the eight LRRK2 kinase inhibitors identified above in FIG. 2. Data are normalized to phosphorylation of LRRK1 and LRRK2 in the absence of compound. Data represent the mean±S.E.M. from three independent experiments;

FIG. 3c shows representative phosphoimage of LRRK1-mediated MBP phosphorylation in the presence and absence of the eight LRRK2 kinase inhibitors. The kinase inhibitor KN-93, which has no effect on LRRK1 kinase activity, is included as a negative control. Coomassie brilliant blue staining (CBB) is shown as a loading control for MBP. Autoradiographs and blot are representative of three independent experiments;

FIG. 3d shows percent inhibition of LRRK1 kinase activity against MBP by the eight LRRK2 kinase inhibitors identified above in FIG. 2. Data are normalized to phosphorylation of MBP in the absence of compound. Data represent the mean±S.E.M. from three independent experiments. (e) Dose-response curves of the effect of Raf-1 kinase inhibitors on LRRK1-mediated MBP phosphorylation. Data represent the mean±S.E.M. from three independent experiments;

FIGS. 4a-4h (a) Quantification of neuronal injury, normalized to the number of viable neurons transacted with eGFP in three experiments. ***P<0.001 and *P<0.05 by ANOVA compared to eGFP control. ***P<0.001 by ANOVA compared to LRRK2 G2019S. *P<0.05 by ANOVA compared to LRRK2 D1994A. Tukey-Kramer post hoc test. Degrees of freedom=21 (total) and F=42.436; (b) Quantification of neuronal injury in the presence or absence LRRK2 kinase inhibitors. ***P<0.001 by ANOVA compared to eGFP control. ***P<0.01 by ANOVA compared to DMSO control. Tukey-Kramer post hoc test. Degrees of freedom=28 (total) and F=47.3152; (c) Quantification of neuronal cell death via TUNEL. ***P<0.001 by ANOVA compared to eGFP control ***P<0.01 by ANOVA compared to LRRK2 G2019S. Neuman-Keuls post hoc test. Degrees of freedom=14 (total) and F=12.4378; (d) TUNEL quantification in the presence or absence of LRRK2 kinase inhibitors. **P<0.01 by ANOVA compared to eGFP control. **P<0.01 by ANOVA compared to DMSO control. Neuman-Keuls post hoc test. Degrees of freedom=20 (total) and F=16.6113; (e) LRRK2 and GFP immunoblots of striatum and substantia nigra (SN) 2 weeks after intrastriatal infusion of HSV-eGFP, WT LRRK2 (HSV-WT LRRK2), G2019S LRRK2 (HSV-G2019S LRRK2) and G2019S-D1994A LRRK2 (HSV-G2019S-D1994A LRRK2); (f) Substantia nigra tyrosine hydroxylase (TH) immunolabelling 3 weeks after HSV-mediated delivery of eGFP, WT LRRK2, G2019S LRRK2 or G2019S-D1994A LRRK2 in the presence or absence of LRRK2 kinase inhibitors. Scale bar, 500 μm; (g) Tyrosine hydroxylase-positive and NissI-positive cell counts comparing eGFP, WT LRRK2, G2019S LRRK2 or G2019S-D1994A LRRK2. Each bar represents the mean number (±S.E.M., n=8) of tyrosine hydroxylase-positive cells. ***P<0.00.1 by ANOVA compared to eGFP control and WT LRRK2. ***P<0.001 by ANOVA compared to G2019S-D1994A LRRK2. Tukey-Kramer post hoc test. Degrees of freedom=67 (total) and F=6.5115 for NissI staining groups. Degrees of freedom=68 (total) and F=7.1292 for tyrosine hydroxylase staining groups; (h) Tyrosine hydroxylase-positive and NissI-positive cell counts comparing LRRK2 G2019S in the presence or absence of LRRK2 kinase inhibitors. Each bar represents the mean number (±S.E.M., n=8) of tyrosine hydroxylase-positive cells. *P<0.05, **P<0.01 and ***P<0.001 by ANOVA compared to DMSO vehicle control. Tukey-Kramer post hoc test. Degrees of freedom=70 (total) and F=5.6004 for NissI staining groups. Degrees of freedom=88 (total) and F=5.0678 for tyrosine hydroxylase staining groups;

FIGS. 5a-5e demonstrate that LRRK2 kinase inhibition protects against LRRK2-induced neuronal toxicity. Representative photomicrographs of each experimental group. LRRK2 and eGFP constructs were combined in a molar ratio of 10:1, respectively, and transfected by using of Lipofectamine 2000 (Invitrogen) at DIV (day in vitro) 14 into rat primary cortical neuronal cultures. Compounds (0.5 μM) were administrated at the time of transfection and continued until toxicity assessments. On DIV 16, images were collected on a Zeiss Automatic stage with Axiovision 6.0. (a) Low magnified photomicrographs; (b and c) High magnified photomicrographs. Scale bar=50 μm (a) and 10 μm (b and c);

FIGS. 6a and 6b demonstrate that intrastriatal administration of HSV amplicon-mediated delivery of GFP triggers targeted gene expression within dopaminergic neurons of the substantia nigra. (a) Representative fluorescent photomicrographs depicting GFP and TH immunolabeling in the striatum and SN following HSV-mediated delivery of GFP via intrastriatal infusion. The striatal images represent four images that were fused to demonstrate the majority of the striatum in one image; (b) Three weeks following transduction of GFP, 71.25%±5.76 (S.E.M.) (n=4) of TH positive cells in the ipsilateral SN were co-labeled with GFP;

FIGS. 7a and 7b show dopaminergic fiber density after HSV amplicon-mediated delivery of LRRK2 WT and LRRK2 G2019S in the mouse striatum. (a and b) HSV amplicon-mediated delivery of LRRK2 G2019S triggers a significant loss of dopaminergic fiber density in the rodent striatum. Each bar represents the mean number (±S.E.M., n=8) of TH immunolabeling measured in four sections through the ipsilateral caudate putamen. ***p<0.001 by ANOVA compared to the other groups, Neuman-Keuls post hoc test. Degree of freedom=39 (total) and F=12.248. Scale bar=200 μm;

FIGS. 8a and 8b show that HSV amplicons do not trigger microglia activation in the ipsilateral striatum and substantia nigra. (a and b) Isolectin B4 (ILB4) immunolabeling in the ipsilateral striatum and substantia nigra pars compacts (SNc) following HSV amplicon-mediated delivery of either GFP, LRRK2 WT, or LRRK2 G2019S to animals receiving twice daily injections of either GW5074 (2.5 mg/kg, i.p.) or its vehicle DMSO. Each bar represents the mean number (±S.E.M., n=8) of ILB4-positive cells counted across four sections. ***p<0.001 by ANOVA compared to the other groups. Neuman-Keuls post hoc test. Degree of freedom=39 (total) and F=35.095 (Striatum). Degree of freedom=39 (total) and F=38.622 (SN). Scale bar=100 μm; and

FIGS. 9a-9c show the results from the transgenic nematode model. In FIG. 9a, isogenic worm strains expressing LRRK2 G2019S in dopaminergic (DA) neurons display neurodegeneration. At the 7 day-old stage, most worms are missing a few anterior DA neurons. Note the absence of 1 ADE neuron in LRRK2 G2019S expressing worms. In FIG. 9b, treatments with compounds GW5074 and Sorafenib protect worms from neurodegeneration where worms display all 4 CEP (arrowheads) and 2 ADE neurons (arrows). In FIG. 9c, worm population analysis revealed that 67%, 66% and 62% of worms treated with 25 μM and 10 μM compound GW5074, as well as, 25 μM compound Sorafenib were wild type (presence of 4 CEP (arrowheads) and 2 ADE neurons (arrows) as shown in FIG. 9b, compared with only 48% of worms without the LRRK2 kinase inhibitors.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Inhibitors of Leucine-Rich Repeat Kinase-2

Leucine-rich repeat kinase-2 (LRRK2) mutations are a common cause of Parkinson's disease. The presently disclosed subject matter identifies inhibitors of LRRK2 kinase that are protective in in vitro and in vivo models of LRRK2-induced neurodegeneration. The presently disclosed subject matter establishes that LRRK2-induced degeneration of neurons in vivo is kinase dependent and that LRRK2 kinase inhibition provides a potential new neuroprotective paradigm for treating Parkinson's disease.

### A. LRRK2 Inhibitors

In some embodiments, the presently disclosed subject matter provides a method for inhibiting a leucine-rich repeat kinase-2 (LRRK2) kinase, the method comprising contacting an LRRK2 kinase with a compound of formulae (I-VII):

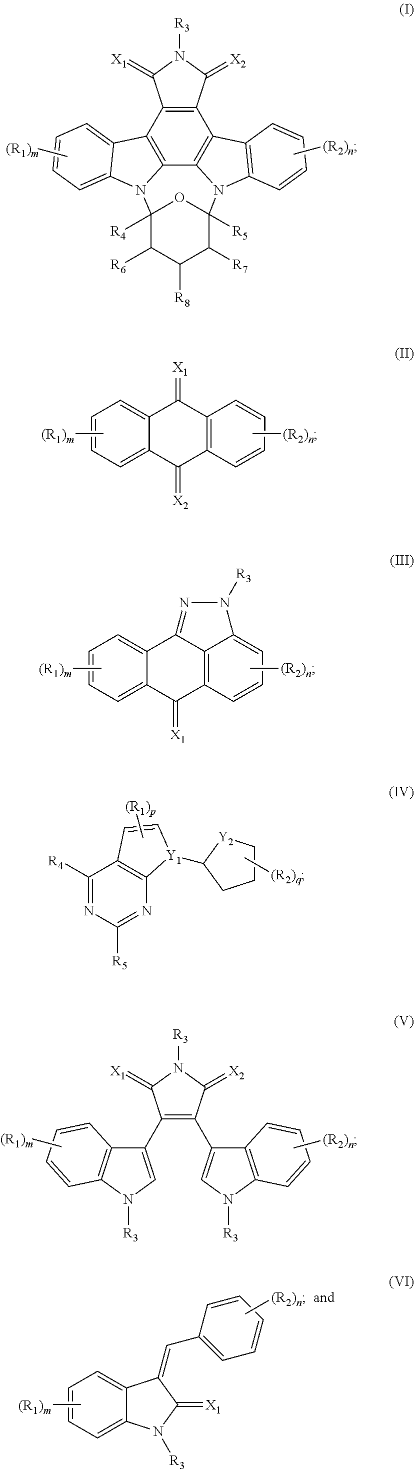

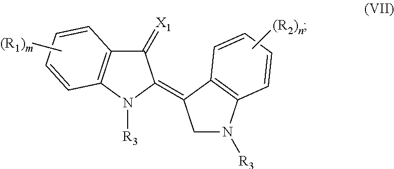

wherein p is an integer selected from the group consisting of 0, 1 and 2; q is an integer selected from the group consisting of 0, 1, 2, and 3: m and n are each an integer independently selected from the group consisting of 0, 1, 2, 3, and 4; each (=X₁) and (=X₂) can be present or absent and, when present, each X₁ and X₂ is independently selected from the group consisting of O, S, CR₉R₁₀, and NR₁₁, wherein R₉, R₁₀, and R₁₁ are each independently selected from the group consisting of H, substituted or unsubstituted alkyl, substituted or substituted heteroalkyl, substituted or substituted alkenyl, alkynyl, substituted or unsubstituted cycloalkyl, substituted or unsubstituted cycloheteroalkyl, substituted or unsubstituted aryl, substituted or unsubstituted heteroaryl, aralkyl, hydroxyl, —COR₁₂, —COOR₁₃, —OR₁₄, wherein R₁₂, R₁₃, and R₁₄ are each independently selected, from the group consisting of H, substituted or unsubstituted alkyl, substituted or substituted heteroalkyl, substituted or substituted alkenyl, alkynyl, substituted or unsubstituted cycloalkyl, substituted or unsubstituted cycloheteroalkyl, substituted or unsubstituted aryl, substituted or unsubstituted heteroaryl; Y₁ is selected from the group consisting of N, and CR₉, wherein R₉ is as defined above; Y₂ is selected from the group consisting of O, S, CR₉R₁₀, and NR₁₁, wherein R₉, R₁₀, and R₁₁ are as defined above; each R₁, R₂, R₄, R₅, R₆, R₇, and R₈ is independently selected from the group consisting of substituted or unsubstituted alkyl, substituted or substituted heteroalkyl, substituted or substituted alkenyl, alkynyl, substituted or unsubstituted cycloalkyl, substituted or unsubstituted cycloheteroalkyl, substituted or unsubstituted aryl, substituted or unsubstituted heteroaryl, arylalkyl, arylalkynyl, alkoxyl, acyloxyl, aryloxyl, arylalkyloxyl, cycloalkylalkyloxyl, cycloalkyloxyl, alkoxyalkyl, alkoxyalkoxyl, aminoalkoxyl, mono- or di-alkylaminoalkoxyl, alkoxycarbonyl, carboxyl, halo, amino, alkylamino, acylamino, arylamino, sulfonyl, arylmercapto, alkylmercapto, hydroxyl, hydroxyalkyl, hydroxycycloalkyl, alkoxycycloalkyl, aminoalkyl, alkylaminoalkyl, cyano, nitro, CF₃, —COR₁₂, —COOR₁₃, and —OR₁₄, wherein R₁₂, R₁₃, and R₁₄ are as defined above; R₃ is selected from the group consisting of H, substituted or unsubstituted alkyl, substituted or substituted heteroalkyl, substituted or substituted alkenyl, alkynyl, substituted or unsubstituted cycloalkyl, substituted or unsubstituted cycloheteroalkyl, substituted or unsubstituted aryl, substituted or unsubstituted heteroaryl, aralkyl, hydroxyl, —COR₁₂, —COOR₁₃, —OR₁₄, wherein R₁₂, R₁₃, and R₁₄ are as defined above; and stereoisomers, prodrugs, and pharmaceutically acceptable salts thereof.

In some embodiments, the compound of formula (I) has the following structure:

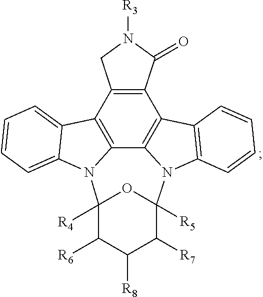

wherein R₃ is selected from the group consisting of H, substituted or unsubstituted alkyl, hydroxyl, and alkoxyl; and R₄, R₅, R₆, R₇, and R₈ are each independently selected from the group consisting of H, substituted or unsubstituted alkyl, alkoxyl, and aminoalkyl.

In particular embodiments, the compound of formula (I) has the following structure:

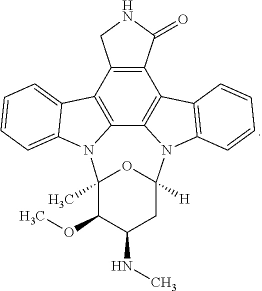 [SMILES: CN[C@@H]1C[C@H]2O[C@@](C)([C@@H]1OC)n1c3ccccc3c3c4c(c5c6ccccc6n2c5c31)C(=O)NC4]

In some embodiments, the compound of formula (II) has the following structure:

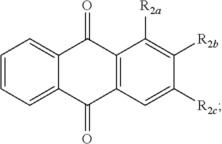

wherein R₂ₐ, R_{2b}, and R_{2c} are selected from the group consisting of hydroxyl, alkoxyl, and —COR₁₂, wherein C₁₂ is selected from the group consisting of H, substituted or unsubstituted alkyl.

In particular embodiments, the compound of formula (II) has the following structure:

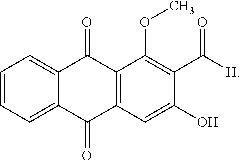

In some embodiments, the compound of formula (III) has the following structure:

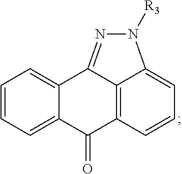

wherein R₃ is selected from the group consisting of H, substituted or unsubstituted alkyl, hydroxyl, and alkoxyl.

In particular embodiments, the compound of formula (III) has the following structure:

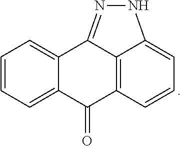 [SMILES: O=C1c2ccccc2-c2n[nH]c3cccc1c23]

In some embodiments, the compound of formula (IV) has the following structure:

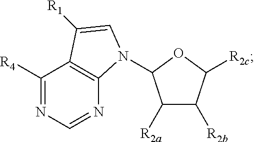

wherein R₁ is halo; R₂ₐ, R_{2b}, and R_{2c} are each independently selected from the group consisting of H, alkyl or unsubstituted alkyl, hydroxyl, alkoxyl, and hydroxyalkyl; and R₄ is selected from the group consisting of H, alkyl or unsubstituted alkyl, hydroxyl, alkoxyl, and amino.

In particular embodiments, the compound of formula (IV) has the following structure:

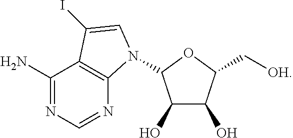

In some embodiments, the compound of formula (V) has the following structure:

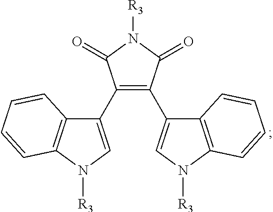

wherein each R₃ is independently selected from the group consisting of H, substituted or unsubstituted alkyl, and substituted or substituted heteroalkyl.

In particular embodiments, the compound of formula (V) is selected from the group consisting of:

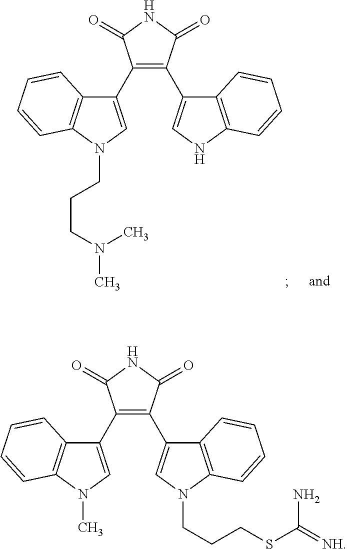 [SMILES: CN(C)CCCn1cc(C2=C(c3c[nH]c4ccccc34)C(=O)NC2=O)c2ccccc21 ; Cn1cc(C2=C(c3cn(CCCSC(=N)N)c4ccccc34)C(=O)NC2=O)c2ccccc21]

In some embodiments, the compound of formula (VI) has the following structure:

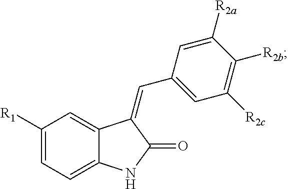

wherein R₁ is halo; and R₂ₐ, R_{2b}, and R_{2c} are each independently selected from the group consisting of H, substituted or unsubstituted alkyl, hydroxyl, alkoxyl, and halo.

In particular embodiments, the compound of formula (VI) has the following structure:

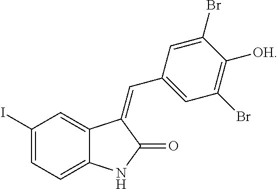 [SMILES: O=C1Nc2ccc(I)cc2/C1=C/c1cc(Br)c(O)c(Br)c1]

In some embodiments, the compound of formula (VII) has the following structure:

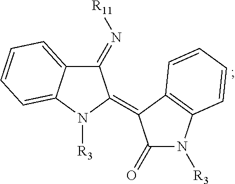

wherein each R₃ is independently selected from the group consisting of H, substituted or unsubstituted alkyl, hydroxyl, and alkoxyl; and R₁₁ is selected from the group consisting of H, substituted or unsubstituted alkyl, hydroxyl, and alkoxyl.

In particular embodiments, the compound of formula (VII) has the following structure:

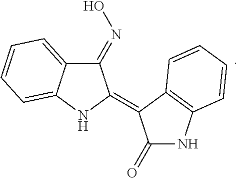 [SMILES: O=C1Nc2ccccc2/C1=C1/Nc2ccccc2/C1=N\O]

### B. Chemical Definitions

While the following terms in relation to compounds of formula (I) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted, for example, with fluorine at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, iso-propyl, cyclopropyl, allyl, vinyl, n-butyl, tert-butyl, ethynyl, cyclohexyl, methoxyl, diethylamino, and the like.

The term “alkyl” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁-C₁₀ means one to ten carbons). In particular embodiments, the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl n-pentyl, sec-pentyl, iso-pentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “heteroalkyl,” by itself or in combination with another term, means, unless otherwise stated, a stable straight or branched chain, or cyclic hydrocarbon group, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH_{2.5}—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)R′, —C(O)NR′, —NR′R″, —OR′, —SR, and/or —SO₂R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkyl group, also as defined above. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₁₋₂₀ inclusive straight or branched hydrocarbon moiety having at least one carbon-carbon double bond by the removal of a single hydrogen atom. Alkenyl groups include, for example, ethenyl (i.e., vinyl), propenyl, butenyl, 1-methyl-2-buten-1-yl, pentenyl, hexenyl, octenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₁₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, heptynyl, and allenyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxo, arylthioxo, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). The term “haloaryl,” however, as used herein, is meant to cover only aryls substituted with one or more halogens.

Where a heteroalkyl, heterocycloalkyl or heteroaryl includes a specific number of members (e.g. “3 to 7 membered”), the term “member” refers to a carbon or heteroatom.

Further, a structure represented generally by the formula:

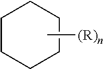

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

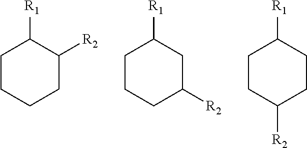

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure. The symbol ( [symbol] ) denotes the point of attachment of a moiety to the remainder of the molecule.

When a named atom of an aromatic ring or a heterocyclic aromatic ring is defined as being “absent,” the named atom is replaced by a direct band. Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such groups. R′, R″,R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl alkoxyl or thioalkoxyl groups, or arylalkyl groups. As used herein, an “alkoxy” or “alkoxyl” group is an alkyl attached to the remainder of the molecule through a divalent oxygen. When a compound of the disclosure includes more than one R group, for example, each of the R groups is independently selected as are each R′, R″, R′″and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CH₃, —C(O)CF₃, —C(O)CH₂OCH₃, and the like.

Similar to the substituents described for alkyl groups above, exemplary substituents for aryl and heteroaryl groups (as well as their divalent derivatives) are varied and are selected from, for example: halogen, —OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″—S(O)R′, —S(O)₂R′, —S(O)₂NR′R″, —NRSO₂R′, —CN and —NO₂, —R′, —N₃, —CH(Ph)₂, fluoro(C₁-C₄)alkoxo, and fluoro(C₁-C₄)alkyl, in a number ranging from zero to the total number of open, valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes: more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}-U-, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ-B-, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C′″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocyclic, heterocyclic, or aromatic heterocyclic group as defined herein). As such, the term “acyl” specifically includes arylacyl groups, such as an acetylfuran and a phenacyl group. Specific examples of acyl groups include acetyl and benzoyl.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, t-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarboxyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an amide group of the formula —CONH₂. “Alkylcarbamoyl” refers to a R′RN—CO— group wherein one of R and R′ is hydrogen and the other of R and R′ is alkyl and/or substituted alkyl as previously described. “Dialkylcarbamoyl” refers to a R′RN—CO— group wherein each of R and R′ is independently alkyl and/or substituted alkyl as previously described.

The term carbonyldioxyl, as used herein, refers to a carbonate group of the formula —O—CO—OR.

“Acyloxy” refers to an acyl-O— group wherein acyl is as previously described.

The term “amino” refers, to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the. group consisting of alkyl groups. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, iso-propylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —(C═O)— group.

The term “carboxyl” refers to the —COOH group. Such groups also are referred to herein as a “carboxylic acid” moiety.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

The term ureido refers to a urea group of the formula —NH—CO—NH₂.

Unless otherwise explicitly defined, a “substituent group,” as used herein, includes a functional group selected from one or more of the following moieties, which are defined herein:

(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and

(B) alkyl, heteroalkyl, cycloalkyl, heterocycloalkyl, aryl, and heteroaryl, substituted with at least one substituent selected from:

(i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and

(ii) alkyl, heteroalkyl, cycloalkyl, heterocycloalkyl, aryl, and heteroaryl substituted with at least one substituent selected from:

(a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and

(b) alkyl, heteroalkyl, cycloalkyl, heterocycloalkyl, aryl, or heteroaryl, substituted with at least one substituent selected from oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “lower substituent” or “lower substituent group,” as used herein means a group selected from all of the substituents described hereinabove for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

A “size-limited substituent” or “size-limited substituent group,” as used herein means a group selected from all of the substituents described above for a “substituent group,” wherein, each, substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

Throughout the specification and claims, a given chemical formula or name shall encompass all tautomers, congeners, and optical- and stereoisomers, as well as racemic mixtures where such isomers and mixtures exist.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those which are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as pharmaceutically acceptable salts. The term “pharmaceutically acceptable salts” is meant to include salts of active compounds which are prepared with relatively nontoxic acids or bases, depending on the particular substituent moieties found on the compounds described herein. Pharmaceutically acceptable salts are generally well known to those of ordinary skill in the art, and may include, by way of example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrates, (e.g. (+)-tartrates, (−)-tartrates or mixtures thereof including racemic mixtures), or teoclate. These salts may be prepared by methods known to those skilled in art. Other pharmaceutically acceptable salts may be found in, for example, Remington: The Science and Practice of Pharmacy (20^{th} ed.) Lippincott, Williams & Wilkins (2000).

Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like.

Also included are salts of amino acids such as arginate and the like, and salts of organic acids like glucuronic or galactunoric acids and the like, see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts. The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

### C. Compositions Comprising LRRK2 Inhibitors of Formulae (I-VII)

The presently disclosed subject matter, in some embodiments, provides a pharmaceutical composition including a therapeutically effective amount of one or more of the presently disclosed compounds of formulae (I-VII) alone or in combination with one or more additional therapeutic agents in admixture with a pharmaceutically acceptable carrier. One of skill in the art will recognize that the pharmaceutical compositions include the pharmaceutically acceptable salts of the compounds described above.

Pharmaceutical compositions suitable for use in the present disclosure include compositions wherein the active ingredients are contained in a therapeutically effective amount to achieve its intended purpose. Determination of the therapeutically effective amounts is well within the capability of those skilled in the art, especially in light of the detailed disclosure provided herein. In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the composition of the encapsulating matrix, the diagnosis or progression of a particular disease state or condition, and the like.

The compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the subject to be treated, the body weight of the subject to be treated, and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone), if desired, disintegrating agents may be added, such as the cross-linked polyvinylpyrrolidone, agar, or alginic acid or a salt thereof such as sodium alginate.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Further, dragee cores comprising the presently disclosed compositions can be provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

In therapeutic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, the presently disclosed compositions may be formulated into liquid or solid dosage forms and administered systemically or locally. The agents may be delivered, for example, in a timed- or sustained-low release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances such as, saline, preservatives, such as benzyl alcohol, absorption promoters, and fluorocarbons.

### D. Method for Treating a Disorder or Condition by Inhibiting LRRK2 Activity

In some embodiments, the presently disclosed subject matter provides a method for treating a disorder or a condition that can be treated by inhibiting LRRK2 activity in a subject in need of treatment thereof, the method comprising administering to the subject a therapeutically effective amount of a compound of formulae (I-VII).

Accordingly, in some embodiments, the presently disclosed subject matter provides a method for treating a disorder or a condition that can be treated by inhibiting LRRK2 activity in a subject in need of treatment thereof, the method comprising administering to the subject a therapeutically effective amount of a compound of formulae (I-VII):

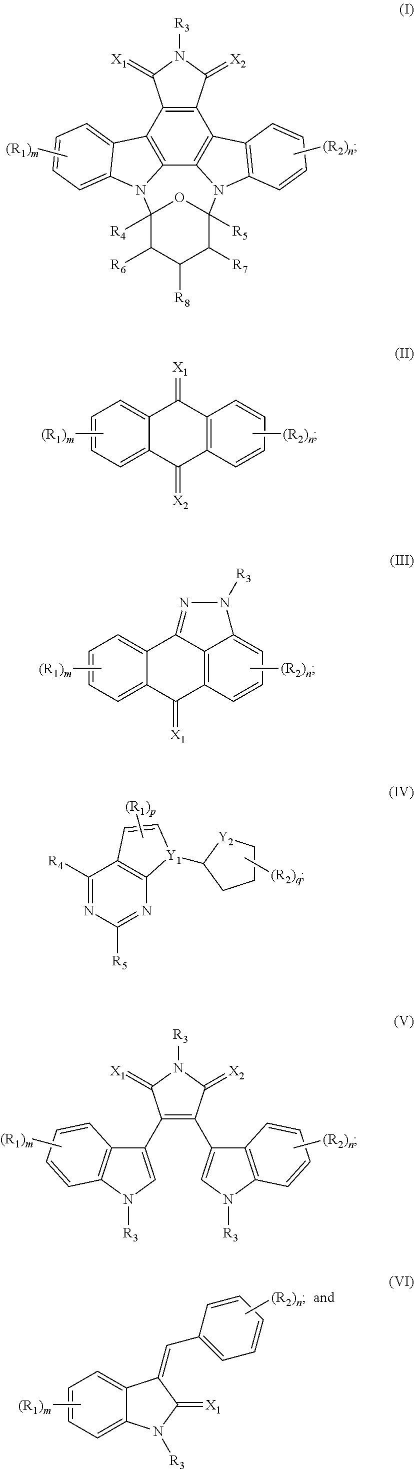

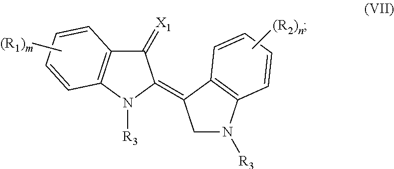

wherein p is an integer selected from the group consisting of 0, 1, and 2; q is an integer selected from the group consisting of 0, 1, 2, and 3: m and n are each an integer independently selected from the group consisting of 0, 1, 2, 3, and 4; each (=X₁) and (=X₂can be present or absent and, when present, each X₁ and X₂ is independently selected from the group consisting of O, S, CR₉R₁₀, and NR₁₁, wherein R₉, R₁₀, and R₁₁ are each independently selected from the group consisting of H, substituted or unsubstituted alkyl, substituted or substituted heteroalkyl, substituted or substituted alkenyl, alkynyl, substituted or unsubstituted cycloalkyl, substituted or unsubstituted cycloheteroalkyl, substituted or unsubstituted aryl, substituted or unsubstituted heteroaryl, aralkyl, hydroxyl, —COR₁₂, —COOR₁₃, —OR₁₄, wherein R₁₂, R₁₃, and R₁₄ are each independently selected from the group consisting of H, substituted or unsubstituted alkyl, substituted or substituted heteroalkyl, substituted or substituted alkenyl, alkynyl, substituted or unsubstituted cycloalkyl, substituted or unsubstituted cycloheteroalkyl, substituted or unsubstituted aryl, substituted or unsubstituted heteroaryl; Y₁ is selected from the group consisting of N, and CR₉, wherein R₉ is as defined above; Y₂ is selected from the group consisting of O, S, CR₉R₁₀, and NR₁₁, wherein R₉, R₁₀, and R₁₁ are as defined above; each R₁, R₂, R₄, R₅, R₆, R₇, and R₈ is independently selected from the group consisting of substituted or unsubstituted alkyl, substituted or substituted heteroalkyl, substituted or substituted alkenyl alkynyl, substituted or unsubstituted cycloalkyl, substituted or unsubstituted cycloheteroalkyl, substituted or unsubstituted aryl, substituted or unsubstituted heteroaryl, arylalkyl, arylalkynyl, alkoxyl, acyloxyl, aryloxy, arylalkyloxyl, cycloalkylalkyloxyl, cycloalkyloxyl, alkoxyalkyl, alkoxyalkoxyl, aminoalkoxyl, mono- or di-alkylaminoalkoxyl, alkoxycarbonyl, carboxyl, halo, amino, alkylamino, acylamino, arylamino, sulfonyl, arylmercapto, alkylmercapto, hydroxyl, hydroxyalkyl, hydroxycycloalkyl, alkoxycycloalkyl, aminoalkyl, alkylaminoalkyl, cyano, nitro, CF₃, —COR₁₂, —COOR₁₃, and —OR₁₄, wherein R₁₂, R₁₃, and R₁₄ are as defined above; R₃ is selected from the group consisting of H, substituted or unsubstituted alkyl, substituted or substituted heteroalkyl, substituted or substituted alkenyl, alkynyl, substituted or unsubstituted cycloalkyl, substituted or unsubstituted cycloheteroalkyl, substituted or unsubstituted aryl, substituted or unsubstituted heteroaryl, aralkyl, hydroxyl, —COR₁₂, —COOR₁₃, —OR₁₄, wherein R₁₂, R₁₃, and R₁₄ are as defined above; and stereoisomers, prodrugs, and pharmaceutically acceptable salts thereof.

In some embodiments, the compound of formula (I) has the following structure:

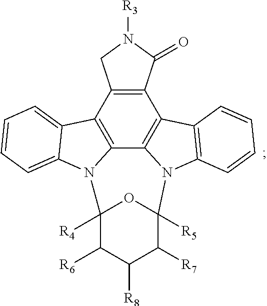

wherein R₃ is selected from the group consisting of H, substituted or unsubstituted alkyl, hydroxyl, and alkoxyl; and R₄, R₅, R₆, R₇, and R₈ are each independently selected from the group consisting of H, substituted or unsubstituted alkyl, alkoxyl, and aminoalkyl.

In particular embodiments, the compound of formula (I) has the following structure:

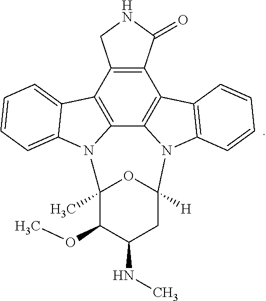 [SMILES: CN[C@@H]1C[C@H]2O[C@@](C)([C@@H]1OC)n1c3ccccc3c3c4c(c5c6ccccc6n2c5c31)C(=O)NC4]

In some embodiments, the compound of formula (II) has the following structure:

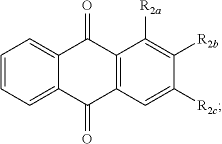

wherein R₂ₐ, R_{2b}, and R_{2c} are selected from the group consisting of hydroxyl, alkoxyl, and —COR₁₂, wherein C₁₂ is selected from the group consisting of H, substituted or unsubstituted alkyl.

In particular embodiments, the compound of formula (II) has the following structure:

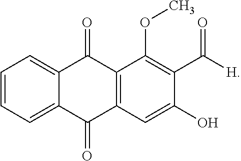

In some embodiments, the compound of formula (III) has the following structure:

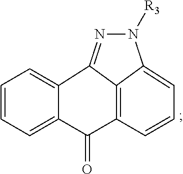

wherein R₃ is selected from the group consisting of H, substituted or unsubstituted alkyl, hydroxyl, and alkoxyl.

In particular embodiments, the compound of formula (III) has the following structure:

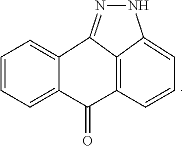 [SMILES: O=C1c2ccccc2-c2n[nH]c3cccc1c23]

In some embodiments, the compound of formula (IV) has the following structure:

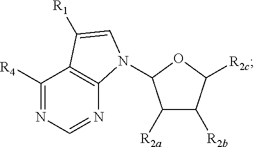

wherein R₁ is halo; R₂ₐ, R_{2b}, and R_{2c} are each independently selected from the group consisting of H, alkyl or unsubstituted alkyl, hydroxyl, alkoxyl, and hydroxyalkyl; and R₄ is selected from the group consisting of H, alkyl or unsubstituted alkyl, hydroxyl, alkoxyl, and amino.

In particular embodiments, the compound of formula (IV) has the following structure:

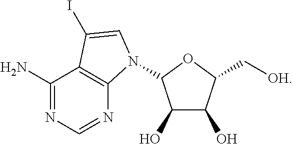

In some embodiments, the compound of formula (V) has the following structure:

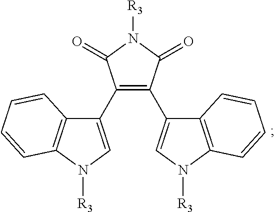

wherein each R₃ is independently selected from the group consisting of H, substituted or unsubstituted alkyl, and substituted or substituted heteroalkyl.

In particular embodiments, the compound of formula (V) is selected from the group consisting of:

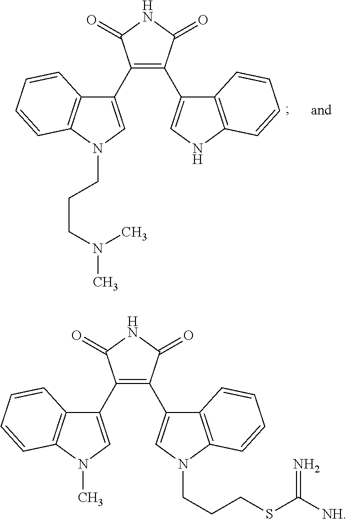

In some embodiments, the compound of formula (VI) has the following structure:

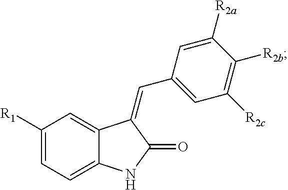

wherein R₁ is halo; and R₂ₐ, R_{2b}, and R_{2c} are each independently selected from the group consisting of H, substituted or unsubstituted alkyl, hydroxyl, alkoxy, and halo.

In particular embodiments, the compound of formula (VI) has the following structure:

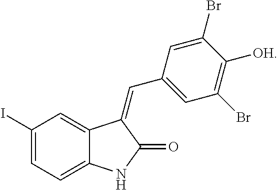 [SMILES: O=C1Nc2ccc(I)cc2/C1=C/c1cc(Br)c(O)c(Br)c1]

In some embodiments, the compound of formula (VII) has the following structure:

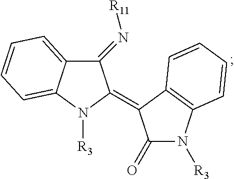

wherein each R₃ is independently selected from the group consisting of H, substituted or unsubstituted alkyl, hydroxyl, and alkoxyl; and R₁₁ is selected from the group consisting of H, substituted or unsubstituted alkyl, hydroxyl, and alkoxyl.

In particular embodiments, the compound of formula (VII) has the following structure:

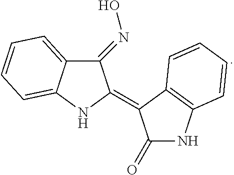 [SMILES: O=C1Nc2ccccc2/C1=C1/Nc2ccccc2/C1=N\O]

The presently disclosed methods treat, prevent, delay the onset or progression of, or alleviate symptoms of a disorder or condition that can be treated by inhibiting LRRK2 activity in a subject in need of treatment thereof by administering to the subject an effective amount of a compound of formulae (I-VII).

In some embodiments, the disorder or condition is a neurodegenerative disease. Examples of neurodegenerative disorders include, but are not limited to, Alexander's disease, Alper's disease, Alzheimer's disease, amyotrophic lateral sclerosis, ataxia telangiectasia. Batten disease, bovine spongiform encephalopathy, Canavan disease, Cockayne syndrome, corticobasal degeneration, Creutzfeldt-Jakob disease, Huntington's disease, HIV-associated dementia, Kennedy's disease, Krabbe's disease, lewy body dementia, Machado-Joseph disease, multiple sclerosis, multiple system atrophy, narcolepsy, neuroborreliosis, Parkinson's disease, Pelizaeus-Merzbacher Disease, Pick's disease, primary lateral sclerosis, prion diseases, Refsum's disease, Sandhoff's disease, Schilder's disease, subacute combined degeneration of spinal cord secondary to pernicious anaemia, schizophrenia, spinocerebellar ataxia, spinal muscular atrophy, Steele-Richardson-Olszewski disease, and tabes dorsalis.

In particular embodiments, the disorder or condition comprises Parkinson's disease or a Parkinson-plus syndrome. Parkinson-plus syndromes include multiple system atropy (MSA) and progressive supranuclear party (PSP). Parkinson's disease can present in several forms, including, but not limited to sporadic Parkinson's disease, a familial form of Parkinson's disease, autosomal recessive early-onset Parkinson's disease, or post-encephalitic Parkinson's disease. The main symptoms of Parkinson's disease are tremor, rigidity of the limbs and trunk, akinesia, bradykinesia and postural abnormalities. A therapeutically effective amount which, when administered to a subject having Parkinson's disease, or a Parkinson-plus syndrome, ameliorates or lessens the severity of one or more of the symptoms of the disease.

In some embodiments, the disease or disorder comprises an autoimmune disease, including inflammatory bowel diseases. In particular embodiments, the autoimmune disease is Crohn's disease. For example, it has been shown that LRRK2 is expressed in immune cells and in the mucosa of subjects afflicted with Crohn's disease and that LRRK2 is involved in immune response. Gardet, A., et al., “LRRK2 Is Involved in the IFN-γ Response and Host Response to Pathogens,” J. Immunol. 185:5577-5585 (2010). More particularly, this study showed that LRRK2 expression is enriched in human immune cells and LRRK2 expression increased in intestinal tissues upon Crohn's disease inflammation. Id. Accordingly, the presently disclosed LLRK2 inhibitors of formulae (I-VII) can be used to treat Crohn's disease.

An “effective amount” of an active agent refers to the amount of the active agent sufficient to elicit a desired biological response. As will be appreciated by one of ordinary skill in the art, the absolute amount of a particular agent that is effective can vary depending on such factors as the desired biological endpoint, the agent to be delivered, the therapeutic effect desired, and the like. One of ordinary skill in the art will further understand that an effective amount can be administered in a single dose, or can be achieved by administration of multiple doses.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

### II. Methods of Screening for LRRK2 Inhibitors

The present invention further provides methods of screening for LRRK2 inhibitors. In one embodiment, the screening method comprises monitoring LRRK2 autophosphorylation in the presence and absence of candidate LRRK2 inhibitors. A compound that reduces, prevents or otherwise inhibits LRRK2 autophosphorylation in comparison to autophosphorylation in the absence of such compound (and optionally in comparison to positive and other negative controls) is indicative that the compound is a candidate LRRK2 inhibitor. In another embodiment, the screening method comprises monitoring LRRK2 phosphorylation of another protein, (e.g., myelin basic protein (“MBP”)) in the presence and absence of candidate LRRK2 inhibitors. A compound that reduces, prevents or otherwise inhibits LRRK2 phosphorylation of MBP in comparison to phosphorylation of MBP in the absence of such compound (and optionally in comparison to positive and other negative controls) is indicative that the compound is a candidate LRRK2 inhibitor. The methods of the present invention may further comprise monitoring LRRK1 autophosphorylation and phosphorylation of MBP in the presence and absence of candidate LRRK2 inhibitors to determine specificity of such inhibitors

The present invention provides additional in vitro methods of screening for LRRK2 inhibitors. More specifically, the present invention provides methods for determining whether LRRK2 inhibition would attenuate neurotoxicity induced by LRRK2 overexpression. In particular embodiments, primary cortical neurons are transiently transacted with wild-type and mutant LRRK2 (e.g., G2019S) and neuronal toxicity is monitored in the presence and absence of candidate LRRK2 inhibitors. Compounds that protects against wild-type and/or G2019S neuronal toxicity are identified as putative LRRK2 inhibitors.

### III. Transgenic Animal and Nematode Models

The present invention further relates to transgenic models. More specifically, the present invention relates to transgenic models of human LRRK2 expression. In particular embodiments, the transgenic models of the present invention express a wild-type human LRRK2 protein. Amino acid sequences of wild type human LRRK2 proteins are known in the art. See GenBank Accession NO. NP-940980.

In certain aspects, the transgenic models of the present invention express a mutant human LRRK2 protein. In specific embodiments, the mutation is a substitution including, but not limited, one or more substitutions at the following positions: N551, I723, R1398, R1441, R1514, P1542, R1628, M1646, S1647, M1869, G2019, G2385 or T2397. In more specific embodiments, the substitution mutation may include, but is not limited, to the following: N551K, I723V, R1398H, R1441C, R1441G, R1441H, R1514Q, P1542S, R1628P, M1646T, S1647T, M1869T, G2019S, G2385R or T2397M. In a specific embodiment, the mutation is at the G2019 position. In a more specific embodiment, the mutation is G2019S.

The transgenic animals of the present invention, which express a mutant human LRRK2 protein, exhibit one or more cardinal phenotypes of Parkinson's disease including, but not limited to, dopaminergic neuron loss, retinal degeneration, locomotor dysfunction and premature mortality.

In one aspect, the present invention provides transgenic animal models. The term “animal” refers to any animal (e.g., a mammal) including, but not limited to, humans, non-human primates, rodents (e.g., mice, rats, etc.), and the like. In particular embodiments, the present invention comprises a transgenic mouse. The term “transgenic” is used in its ordinary sense, includes germline and non-germline expression of transgenes in animals, and further includes the expression of a gene in one or more cells of an animal.

In particular embodiments, the present invention provides a transgenic non-human mammal whose genome comprises a human wild-type LRRK2 gene. The present invention further provides a transgenic non-human mammal whose genome comprises a human mutant LRRK2 gene, wherein expression of the gene creates a Parkinson's-like phenotype. In particular embodiments, the mutant human LRRK2 gene is a G2019S mutation. The Parkinson's-like phenotype may comprise any known phenotype of Parkinson's disease including, but not limited to, dopaminergic neuron loss, retinal degeneration, locomotor dysfunction and premature mortality. In particular embodiments, the non-human mammal is a rodent. In a specific embodiment, the rodent is a mouse.

The term “genome” refers to the entire DNA complement of an organism including nuclear DNA (chromosomal or extrachromosmal DNA) as well as mitochondrial DNA. In certain embodiments, the phrase “whose genome comprises” refers to the stable integration of a gene into the germline cells of a non-human mammal or a nematode. In other embodiments, the phrase refers to the presence of a gene in one or more cells of the non-human mammal including, for example, the expression of a mutant LRRK2 gene via the Herpes Simplex Virus Amplicon expression and delivery platform described herein.

More particularly, a transgenic non-human mammal of the present invention may be a Herpes Simplex Virus (“HSV”) amplicon-based model of LRRK2 that exhibits Parkinson's-like phenotypes. More specifically, the transgenic non-human mammal may be an HSV amplicon-based model of LRRK2 dopaminergic neurotoxicity. As described herein, the present invention further provides methods for generating such transgenic non-human mammals.

The transgenic mammals of the present invention may be used to screen for compounds that modulate LRRK2 activity. More specifically, as described herein, the transgenic mammals may be used to test whether candidate inhibitors of LRRK2 rescue or protect against one or more Parkinson's-like phenotypes including, but not limited to dopaminergic neuron loss, retinal degeneration, locomotor dysfunction and premature mortality. In a specific embodiment, the transgenic mammals may be used to test whether a candidate LRRK2 inhibitor is protective against loss of dopamine neurons.

In other embodiments, the present invention provides methods for screening candidate compounds for the ability to modulate LRRK2 activity in a transgenic non-human mammal and reduce changes associated with the Parkinson's-like phenotype induced by LRRK2 transgene expression. The method may comprise exposing the transgenic non-human mammal to an effective amount of a candidate compound to modulate activity of the LRRK2 protein, and determining whether the compound has a significant effect on the Parkinson's-like phenotype of the transgenic non-human mammal as compared to a transgenic non-human mammal expressing wild-type or mutant LRRK2 that was not exposed to the candidate compound. A compound that has an effect on the Parkinson's-like phenotype of the transgenic non-human mammal induced by activity of the expressed LRRK2 protein is identified as a candidate compound for modulating activity of the LRRK2 protein.

In other embodiments, the method may comprise exposing the transgenic non-human mammal to an environmental stressor to accelerate expression of the Parkinson's-like phenotype, exposing the transgenic non-human, mammal to an effective amount of a candidate compound to modulate activity of the LRRK2 protein, and determining whether the compound has a significant effect on the Parkinson's-like phenotype of the transgenic non-human mammal as compared to a transgenic non-human mammal expressing wild-type or mutant LRRK2 that was not exposed to the candidate compound. The environmental stressor may be any known stressor associated with Parkinson's disease, and includes any stressor that accelerates a Parkinson's-like phenotype resulting from LRRK2 expression. Environmental stressors may include, but are not limited to, oxidative stress, insecticides, 1-methyl-4-phenyl-1,2,3,6-tetrahydropyridine, Nitro oxide (NO) donor, and proteosome inhibitors.

In another aspect, the present invention provides transgenic nematode models. In certain embodiments, the nematode belongs to the subgenus Caenorhabditis. In specific embodiments, the nematode is Caenorhabditis elegans (“C. elegans”).

In certain embodiments, the present invention provides a transgenic nematode whose genome comprises a human wild-type LRRK2 gene. The present invention further provides a transgenic nematode whose genome comprises a mutant human LRRK2 gene, wherein expression of the gene creates a Parkinson's-like phenotype. In particular embodiments, the nematode is C. elegans. In other embodiments, the mutant human LRRK2 gene is a G2019S mutation. The Parkinson's-like phenotype comprises any known phenotype of Parkinson's disease including, but not limited to, dopaminergic neuron loss, retinal degeneration, locomotor dysfunction and premature mortality.

In further embodiments, the present invention provides methods for screening candidate compounds for the ability to modulate LRRK2 activity in a transgenic nematode and reduce changes associated with the Parkinson's-like phenotype induced by LRRK2 transgene expression. The method may comprise exposing the transgenic nematode to an effective amount of a candidate compound to modulate activity of the LRRK2 protein, and determining whether the compound has a significant effect on the Parkinson's-like phenotype of the nematode as compared to a nematode expressing wild-type or mutant LRRK2 that was not exposed to the candidate compound. A compound that has an effect on the Parkinson's-like phenotype of the transgenic nematode induced by activity of the expressed LRRK2 protein is identified as a candidate compound for modulating activity of the LRRK2 protein.

In other embodiments, the method may comprise exposing the transgenic nematode to an environmental stressor to accelerate expression of the Parkinson's-like phenotype, exposing the transgenic nematode to an effective amount of a candidate compound to modulate activity of the LRRK2 protein, and determining whether the compound has a significant effect on the Parkinson's-like phenotype of the nematode as compared to a nematode expressing wild-type or mutant LRRK2 that was not exposed to the candidate compound. The environmental stressor may be any known stressor associated with Parkinson's disease, and includes any stressor that accelerates a Parkinson's-like phenotype resulting from LRRK2 expression. Environmental stressors may include, but are not limited to, oxidative stress, insecticides, 1-methyl-4-phenyl-1,2,3,6-tetrahydropyridine, Nitro oxide (NO) donor, and proteasome inhibitors.

### IV. General Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term, “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Cell Culture and Protein Purification

HEK-293FT cells were cultured in Opti-MEM media (Invitrogen) supplemented with 10% PBS. Transfection was performed by using Fugene HD reagent (Roche Applied Science) according to the manufacturer's instruction. For purification of recombinant LRRK1 and LRRK2 proteins, ACTA Purifier (GE Healthcare) FPLC system was used.

### Example 2

### Generation of GST-Tagged LRRK1 and LRRK2 Plasmids

The GST gene was fused to LRRK1 and LRRK2 via PCR using oligonucleotides pair 1 (forward: 5″ cggatccatgtcccctatactcgg-3′ reverse: 5′-tcagtcagtcacgatgcggc-3′) and pair 2 (forward: 5′-tactcgaggtccttttccaaggtccaatgtcccctatactag-3′ reverse: 5′-tactcgagttacgattttggataatcgccacc-3′) for LRRK1 and LRRK2, respectively, using pGEX6P-1 as template. PCR products were subcloned into pCR2.1 by using the TOPO-TA kit (Invitrogen). For LRRK2, the GST gene was digested by XhoI and BamHI and then ligated to pcDNA3.1-hLRRK2). For LRRK1, the GST gene was digested by BamHI and then ligated to pCMV-2XMyc-hLRRK1.

### Example 3

### LRRK2 Purification

HEK-293FT cells transiently transacted with each GST-tagged LRRK1 and LRRK2 plasmid were harvested and lysed with lysis buffer containing 0.5% NP-40, 150 mM NaCl, 50 mM Hepes (pH 7.4), 5 mM EGTA and complete protease inhibitors. Resulting lysates were rotated at 4° C. for 30 min followed by centrifugation at 20,000×g for 15 min. The supernatants were injected into a GSTrap FF column pre-equilibrated with PBS at 0.5. mL/min flow after filtration using a 0.2 μm syringe filter (Corning). The bound proteins were eluted with buffer containing 10 mM GSH (Sigma), 150 mM NaCl, and 50 mM Tris-HCl (pH 8.0).

### Example 4

### GST-4E-BP1 Purification

The GST-4E-BP1 expressing vector was generated using the Invitrogen Gateway LR Clonase System. Briefly, an entry clone containing the 4E-BP1 sequence was reacted with the destination vector, pDEST-15 in the presence of the LR clonase mixture. After the clonase reaction, the mixture was transformed into One Shot BL21 chemically competent E. coli. The transformed cells were grown on ampicillin containing LB agar plate for one day at 37° C. Colonies containing GST-4E-BP1 were isolated. For protein induction, 100 μM IPTG was added to 100 mL culture and incubated for 3 h at 30° C. After IPTG incubation, cells were collected by centrifugation at 3,000×g for 15 min. The pellet was lysed with sonication in the presence of lysis butter containing 0.5% TX-100, 50 mM Tris-HCl (pH 8.0), 100 mM NaCl, 0.5 mM EGTA, 5 mM MgCl₂, 2 mM DTT, and complete protease inhibitors. The lysate was centrifugated at 15,000×g for 15 min and the supernatant was incubated with GSH-sepharose beads for 2 h at 4° C. After incubation, the beads were washed with PBS containing an additional 150 mM NaCl and 0.1% TX-100 three times. The protein was eluted with elution buffer containing Hepes (pH 8.0), 150 mM NaCl, 30% glycerol, and 30 mM glutathione. The eluted proteins were stored at −80° C.

### Example 5

### In Vitro Kinase Assay

The in vitro kinase assay was performed as previously described. West, A. B. et al. Parkinson's disease-associated mutations in leucine-rich repeat kinase 2 augment kinase activity. Proc Natl Acad Sci USA 102, 16842-16847 (2005). Recombinant LRRK2 and MBP or CST-4E-BP1 were combined before the kinase reaction. LRRK2 autophosphorylation is LRRK2 itself phosphorylation and MBP or GST-4E-BP1 phosphorylation is induced by LRRK2. After the kinase reaction, combined LRRK2 and MBP or GST-4E-BP1 were loaded on SDSPAGE and phosphorylated LRRK2 and MBP or GST-4E-BP1 could be detected separately an a single gel. Recombinant proteins and inhibitors were incubated at 30° C. in kinase assay buffer containing 20 mM Hepes (pH 7.4), 5 mM EGTA, and 20 mM β-glycerol phosphate. 0.5 μg MBP (Upstate) or 0.5 μg GST-4B-BP1, 50 μM ATP (Sigma), 20 mM MgCl2, and 1 μCi [γ-32P] ATP (PerkinElmer) was added after 5 min. The reaction was performed at 30° C. to reduce non-specific 32P incorporation for 15 min and terminated by the addition of 5×Laemmili sample buffer. The reactions were then heated at 70° C. for 10 min and resolved by 12% SDS-PAGE. Gels were stained by coomassie brilliant blue after fixation with 10% acetic acid and 40% methanol and then exposed to a Phosphoimager. Data were analyzed by ImageQuant 6.0 software.

### Example 6

### Western Blot Analysis

Reactions or lysates were resolved via SDS-PAGE and transferred to a poly(vinylidine difluoride) membrane. The membrane was blocked with TBST (10 mM Tris-HCl, pH 7.4, 150 mM NaCl, 0.1% Tween 20) containing 5% skim milk and then probed with anti-LRRK2, anti-GST (Invitrogen), anti-GFP (Abeam) and anti-actin (Sigma) antibodies. The immunoblot was then washed and incubated with horseradish peroxidase-linked secondary antibody for 1 h at room temperature, rinsed four times in TBST, and developed with horseradish peroxidase-dependent chemiluminescence reagents (Pierce Biotechnology).

### Example 7

### Primary Rat Cortical Neuronal Culture and Neuronal Viability

Primary cortical neuronal cultures were prepared from gestational day 15 fetal rats as previously described. Gonzalez-Zulueta, M., et al. Manganese superoxide dismutase protects nNOS neurons from NMDA and nitric oxide-mediated neurotoxicity. J Neurosci 18, 2040-2055 (1998). All procedures used in this study were approved by the Johns Hopkins Medical Institute Animal Care Committee. Cell viability assays and DNA fragmentation (TUNEL assay) were performed as previously described. Smith, W. W., et al. Kinase activity of mutant LRRK2 mediates neuronal toxicity. Nat Neurosci 9, 1231-1233 (2006); West, A. B., et al. Parkinson's disease-associated mutations in LRRK2 link enhanced GTP-binding and kinase activities to neuronal toxicity. Hum Mol Genet 16, 223-232 (2007). The cortex was dissected, incubated for 15 min in 0.027% trypsin, and then transferred to modified Eagle's medium (MEM)/10% horse serum/10% fetal bovine serum/2 mM glutamine followed by trituration. Dissociated cells were plated at a density of 3-4×105 cells per well in polyornithine-coated plates. After 4 days the cells were treated with 10 μg of 5-fluoro-2′-deoxyuridine to prevent proliferation of nonneuronal cells. Cells were maintained in MEM/5% horse serum/2 mM glutamine in 8% CO2 incubator. The medium was changed twice weekly. For assessment of LRRK2-mediated neuronal injury, LRRK2 and eGFP constructs were combined in a molar ratio of 10:1, respectively, and transfected into neurons with Lipofectamine 2000 (Invitrogen) at DIV (day in vitro) 14. On DIV 16, images were collected on a Zeiss Automatic stage with Axiovision 6.0. Viable neurons were defined as having at least one smooth extension (neurite) with twice the length of the cell body. The percentage of eGFP-positive injured neurons in each experimental group relative to those neurons transfected with eGFP was calculated.

### Example 8

### Transgenic Nematode Model

Nematodes were maintained following the standard procedures. Brenner, S., 771 GENETICS 71-94 (1974). Isogenic worm strains expressing LRRK2, G2019S, UAXX [baInlX; P_{dat-1}::LRRK2 G2019S, P_{dat-1}::gfp] were generated via microinjection, integrated into the genome and out-crossed three times. Age-synchronized worms were obtained by treating gravid adults with 2% sodium hypochlorite and 0.5M NaOH to isolate embryos. Lewis, J. A., 48 METHODS CELL BIOL. 3-29 (1995). These embryos were treated with indicated concentrations of LRRK2 kinase inhibitors, which were dissolved in 1% DSMO, for 24 h. at 20° C. with gentle shaking. The worms were then washed and transferred into NGM plates seeded with OP50 bacteria, and incubated as 20° C. The six anterior DA neurons (4 CEP and 2 ADE neurons) of 30 animals/trial were examined for neurodegeneration when the animals were 7 days old. A total of 90 animals for each treatment were analyzed (3 trials of 30 animals/trial). If a worm displayed at least one degenerative change (dendrite or axon loss, cell body loss), the animals were scored as exhibiting degenerating neurons. See Hamamichi et al., 105(2) PROC. NATL. ACAD. SCI. USA 728-33 (2008); and Cao et al, 25 J. NEUROSCI. 3801-12 (2005). For each trial, 30 worms were transferred into a 2% agarose pad, immobilized with 2 mM levamisole, and analyzed using Nikon Eclipse E800 epifluorescence microscope equipped with Endow GFP HYQ filter cube (Chrom Technology, Rockingham, Vt.). Images were captured with a Cool Snap CCD camera (Photometrics, Tucson, Ariz.) driven by MetaMorph software (Universal Imaging, West Chester, Pa.).

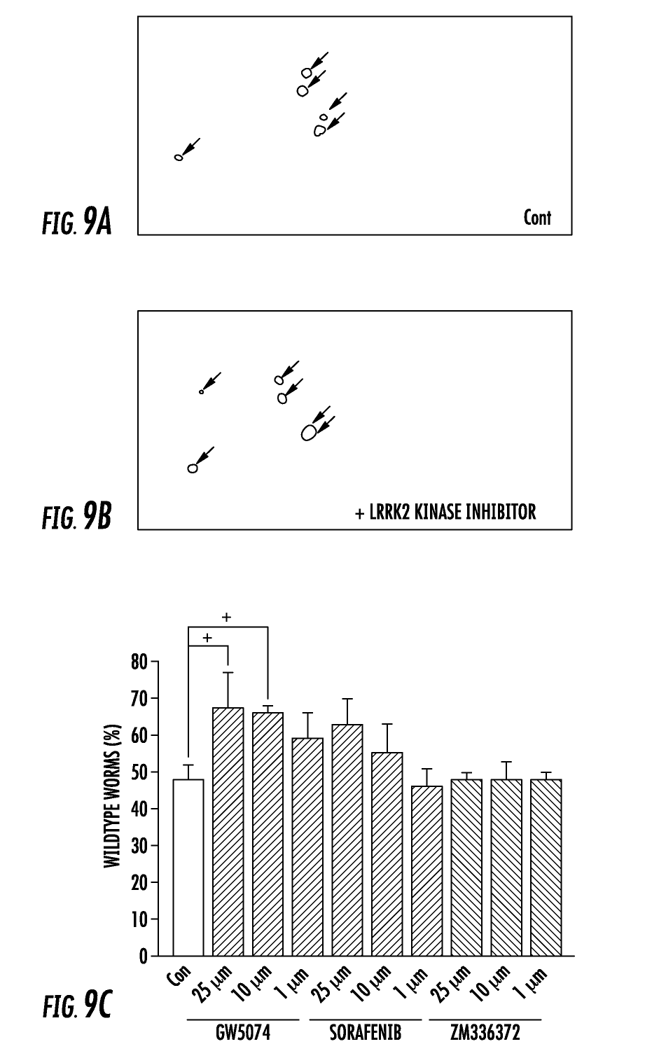

To determine whether the LRRK2 kinase inhibitors protect against LRRK2-induced neurodegeneration in vivo, the LRRK2 kinase inhibitors were tested in the C. elegans model of LRRK2 G2019S-induced neurodegeneration described above. The DA transporter (dat-1) was used to direct expression of LRRK2 G2019S in DA neurons. Expression of LRRK2 G2019S resulted in DA neurodegeneration in 52% of 7-day-old worms analyzed (n=90) after 1% DMSO treatment, indicating that 48% of the population displayed wild type DA neurons (FIGS. 9a and 9b). Treatments with GW5074 (25 and 10 μM) and Sorafenib (25 μM) significantly rescued DA neurodegeneration whereby 67%, 66% and 62% of the same stage animals (n=90 for each treatment) exhibited wild-type neurons (FIGS. 9b and 9c); p<0.05, student's t-test). ZM336372 failed to rescue the DA neurodegeneration in 7 day-old worms (FIG. 9c). These results taken together indicate that inhibition of LRRK2 kinase activity and not LRRK1 or RAF kinase activity protects against LRRK2 neurotoxicity in vitro (primary cortical neuronal cultures; data not shown) and in vivo.

### Example 9

### HSV Amplicon Vector Generation

The HSV amplicon platform was used to generate HSV-LRRK2 expression vectors. Maguire-Zeiss, K. A., et al., HSV vector-mediated gene delivery to the central nervous system. Curr Opin Mol Ther 3, 482-490 (2001). LRRK2 WT, LRRK2 G2019S, and LRRK2 G2019S/D1994A open reading frames were subcloned from pcDNA3.1 to the previously described vector pHSVPrPUC/CMVeGFP. LRRK2 cDNA was amplified using Accuprime polymerase (Invitrogen) using forward and reversed primers that restore the endogenous LRRK2 stop codon sequence and encode flanking KpnI sites and product was inserted into pCR2.1 via TOPO-TA cloning. Inserts were fully sequenced and inserted into pHSVPrPUC/CMVeGFP with a single KpnI digestion. Correct LRRK2 orientation was determined through restriction digest. Expression of native untagged LRRK2 WT, LRRK2 G2019S or LRRK2 G2019S/D1994A was under the transcriptional control of the HSV immediate-early 4/5 (IE4/5) gene promoter, while a separate eGFP expression unit within each amplicon vector was driven by the cytomegalovirus (CMV) immediate-early promoter. Verified plasmids were transiently transfected in HEK-293T cells and LRRK2 expression was validated by Western blot using anti-LRRK2 antibody (Novus 267), prior to amplicon generation. High titer helper free HSV-1 amplicon stocks were generated as previously described. Bowers, W. J., et al., Expression of vhs and VP16 during HSV-1 helper virus-free amplicon packaging enhances titers. Gene Ther 8, 111-120 (2001); Bowers, W. J., et al., Discordance between expression and genome transfer titering of HSV amplicon vectors: recommendation for standardized enumeration. Mol Ther 1, 294-299 (2000); and Jin, B. K., et al., Prolonged in vivo gene expression driven by a tyrosine hydroxylase promoter in a defective herpes simplex virus amplicon vector. Hum Gene Ther 7, 2015-2024(1996).

### Example 10

### HSV Amplicon Delivery of EGFP and LRRK2, Drug Administration, Immunohistochemistry and Stereology

For stereotaxic injection of HSV overexpressing eGFP, LRRK2 WT, LRR2 G2019S, or LRRK2 G2019S/D1994A, experimental procedures were followed according to the guidelines of Laboratory Animal Manual of the National Institute of Health Guide to the Care and Use of Animals. All procedures used in this study were approved by the Johns Hopkins Medical Institute Animal Care Committee and by the Mayo Foundation Institutional Animal Care and Use Committee (IACUC). For a subset of animals, mice were anaesthetized using isoflurane (1%) and placed in a Kopf stereotaxic frame. For nigrostriatal transduction either HSVPrPUC/CMVeGFP, HSV-WT-LRRK2/CMVeGFP, or HSV-G2019S-LRRK2/CMVeGFP amplicon vector (10,000 infectious particles/μL) was injected unilaterally into the striatum (A.P. +1.2, M.L. −1.3, D.V. −3.2) (2 μL/site) at a rate of 0.25 μL/minute via an infusion cannula connected by polyethylene tubing (50 PE) to a 50-μL Hamilton syringe driven by a Harvard pump. Following infusion, the cannula remained in place for four minutes to prevent reflux. For another subset, of animals and in vivo toxicity assessments, six-week-old male C57BL mice (8 animals per each group) (Charles River Laboratories, Inc) were anesthetized with pentobarbital (60 mg/kg). For nigrostriatal transduction either HSVPrPUC/CMVeGFP, HSVLRRK2WT/CMVeGFP, HSV-LRRK2 G2019S/CMVeGFP, or HSV-LRRK2 G2019S/D1994A/CMVeGFP amplicon vectors (10,000 infectious particles/μL) was injected unilaterally into the striatum with an injection cannula (25 gauge, Hamilton) that was applied stereotaxically into the striatum (anteroposterior, −1.2 mm from bregma; mediolateral, 1.3 mm; dorsoventral, 3.2 mm). The infusion was performed at a rate of 0.2 μL/min and wound healing and recovery were monitored after the injection was done. Following infusion, the cannula remained in place for four minutes to prevent reflux. Intrastriatal infusions were chosen in an effort to avoid nonspecific damage of the substantia nigra. Instrastriatal infusions of HSV amplicons lead to robust and sustained expression of the transgene in the ipsilateral substantia nigra. Jin, B. K. et al. Prolonged in vivo gene expression driven by a tyrosine hydroxylase promoter in a defective herpes simplex virus amplicon vector. Hum Gene Ther 7, 2015-2024 (1996).

Three weeks after administration with DMSO containing GW5074, indirubin, or indirubin-3′-monooxime (2.5 mg/kg, i.p. twice daily injection), animals were perfused with PBS followed by 4% paraformaldehyde. Brains were post-fixed with 4% paraformaldehyde, cryoprotected in 30% sucrose, and processed for immunohistochemistry. Forty-micrometer coronal sections were cut throughout the brain including striatum or SN. Every 4th section was taken for further analysis. For tyrosine hydoxylase (TH) staining, sections were reacted with a 1:1000 dilution of rabbit polyclonal anti-TH (Novus) and visualized with biotinylated goat anti-rabbit IgG, followed by streptavidin-conjugated horseradish peroxidase (HRP) (Vectastain ABC kit, Vector Laboratories, Burlingame, Calif.). Positive immunostaining was visualized with 3,3′-diaminobenzidine (DAB, Sigma) after reaction with hydrogen peroxide (DAB kit, Vector Laboratories). Stained sections were mounted onto slides and counterstained with thionin for NissI substance. Total numbers of TH−, and NissI-stained neurons in SNc were counted using the Optical Fractionator probe of Stereo Investigator software (MicroBrightfield, Williston, Vt.), For co-immunostaining, coronal sections were incubated with rabbit polyclonal anti-TH and mouse monoclonal anti-GFP (Abcam) and donkey anti-rabbit-Cy3, donkey anti-mouse-Cy2 were used for microscopic analysis. Two weeks post-intrastriatal injection, the striatum and corresponding SN were surgically dissected and subjected to western blot analysis to validate whether the viruses were expressing LRRK2 and eGFP in vivo and that HSV is retrogradely transported to the SN. For immunoblot analysis, the brain tissues were homogenized in lysis buffer consisting of 10 mM Tris-HCl, pH 7.4, 150 mM NaCl, 5 mM EDTA, 0.5% Nonidet NP-40, 10 mM Na-β-glycerophosphate, Phosphate phosphatase Inhibitor Cocktail I and II (Sigma), and Complete Protease Inhibitor Mixture (Roche). The homogenate was centrifuged at 20,000×g for 20 min, and the resulting supernatants were collected. Protein concentration was quantified by the BCA kit (Pierce) with BSA standards. Western blot analysis was performed with anti-LRRK2, anti-GFP, and anti-actin antibodies.

### Example 11

### Determination of IC₅₀ Value

The IC₅₀ was defined as the concentration of a compound needed to inhibit phosphorylation by 50% of control activity (i. e., in the absence of compound). To calculate the IC₅₀ value, a dose-response curve was constructed and non-linear regression analysis using the equation for a sigmoid plot.

### Example 12

### Statistical Analysis

One-way analysis of variance (ANOVA) was used followed by Tukey-Kramer or Newman-Keuls post-hoc test. Data represent mean±S.D. or S.E.M., p<0.05 was considered statistically significant.

### Example 13

### Results

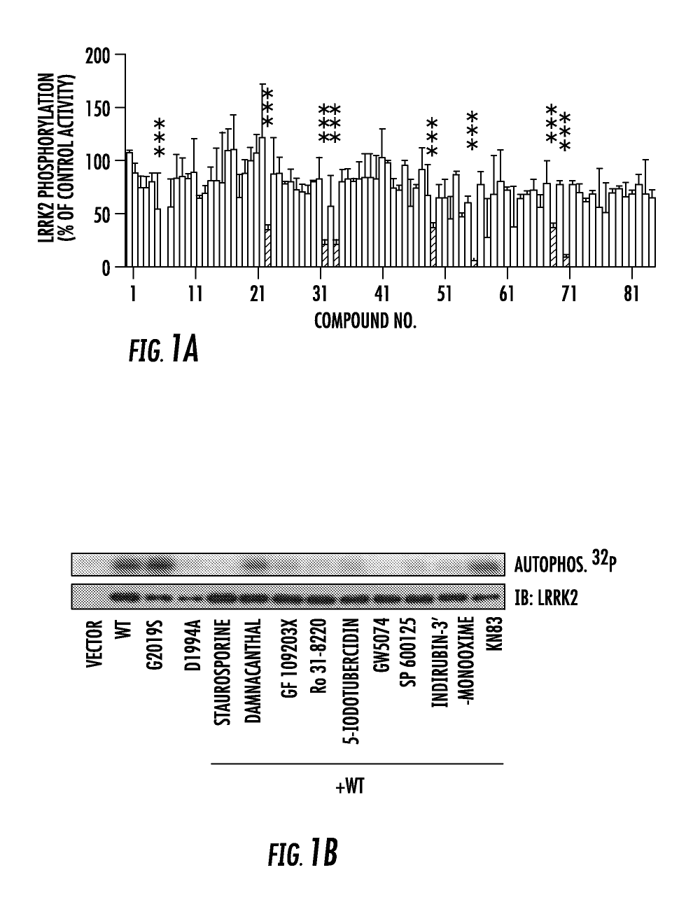

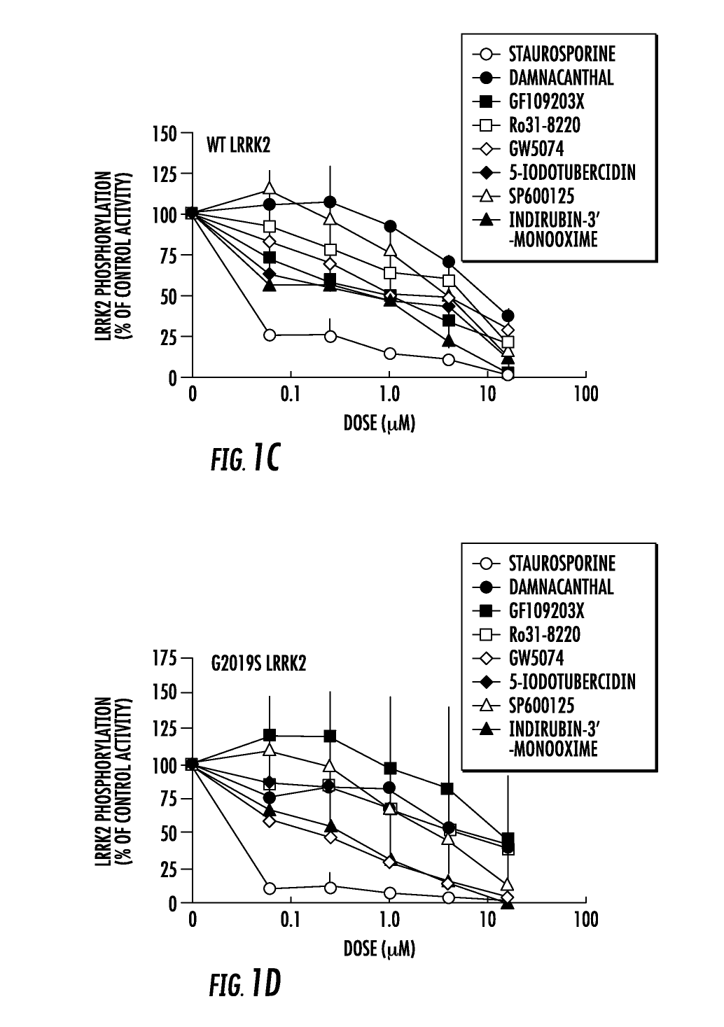

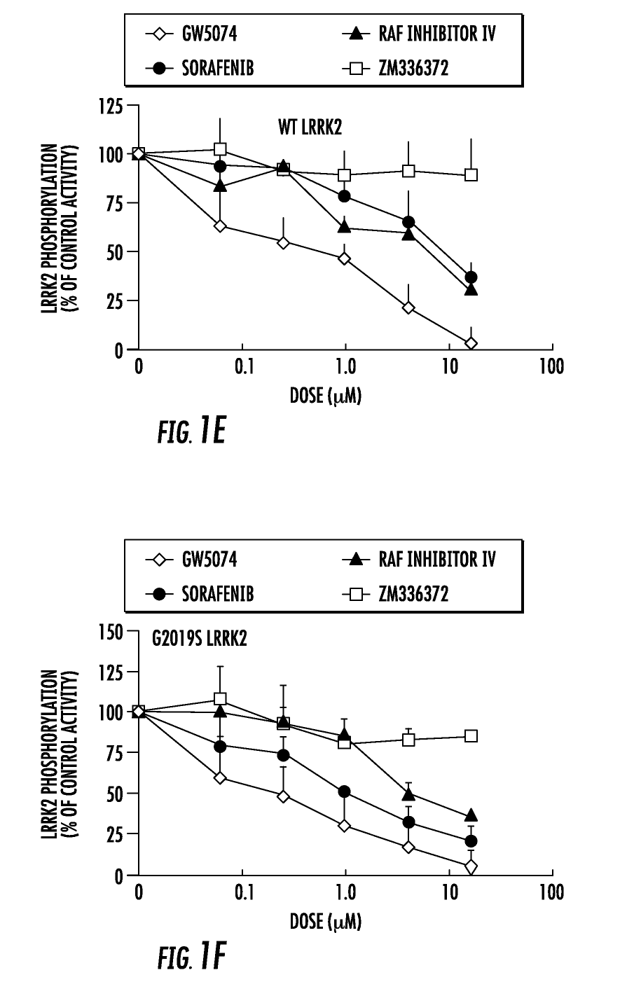

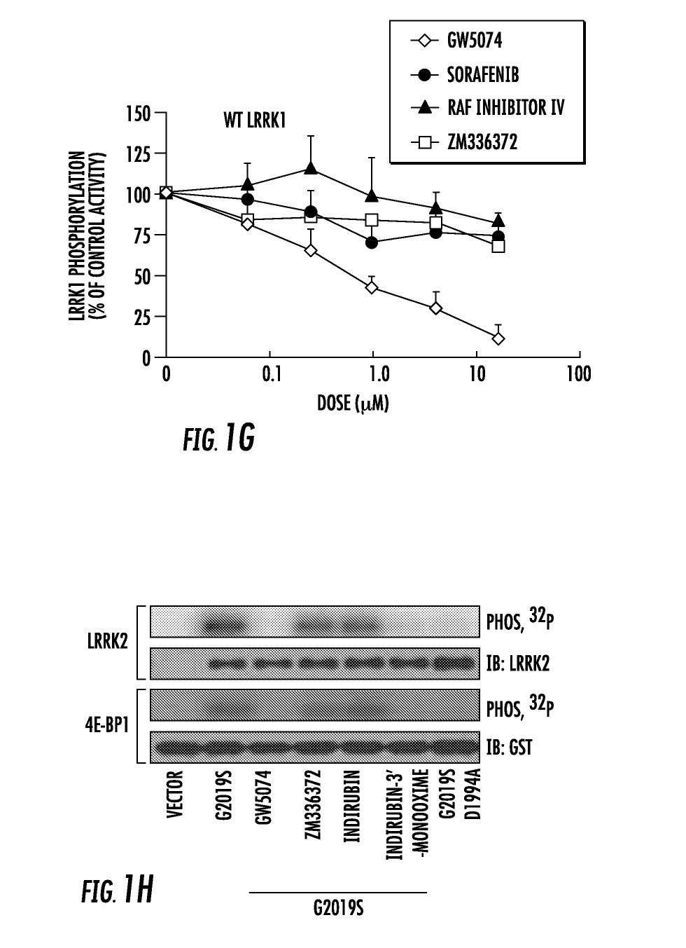

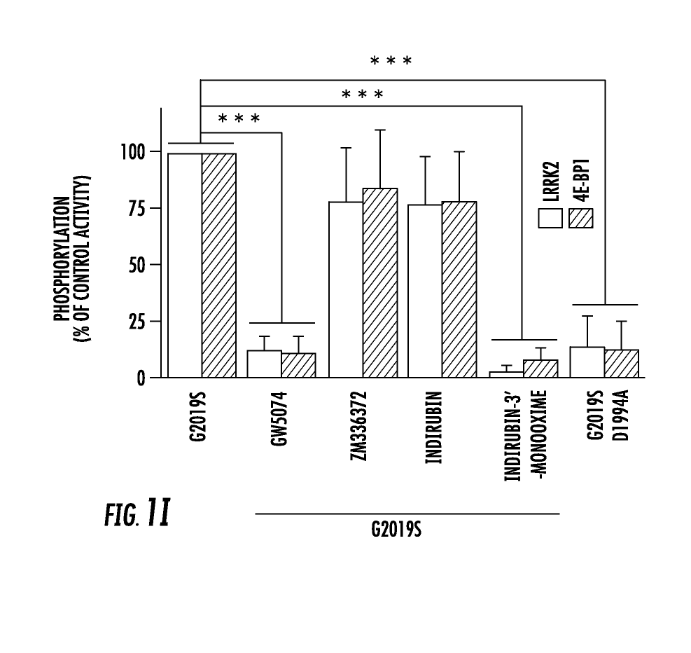

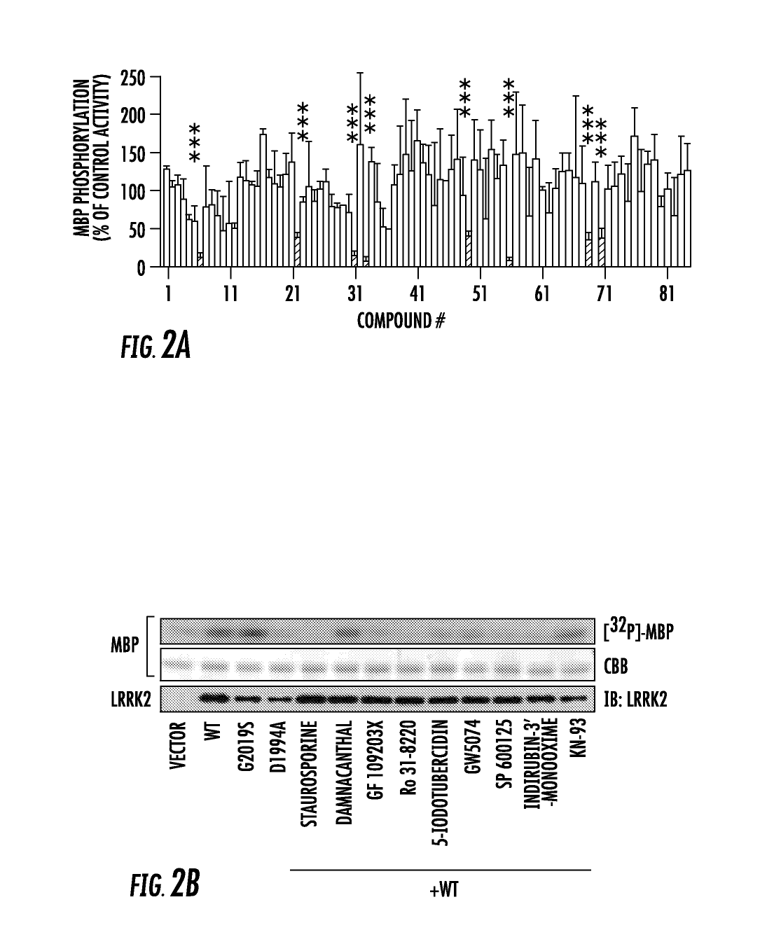

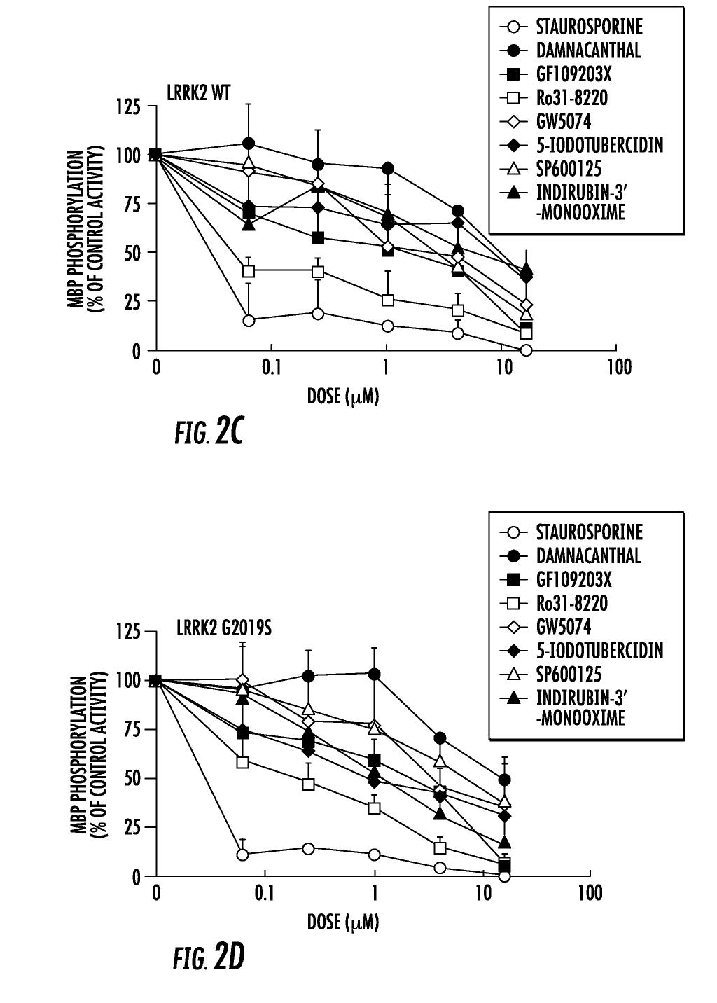

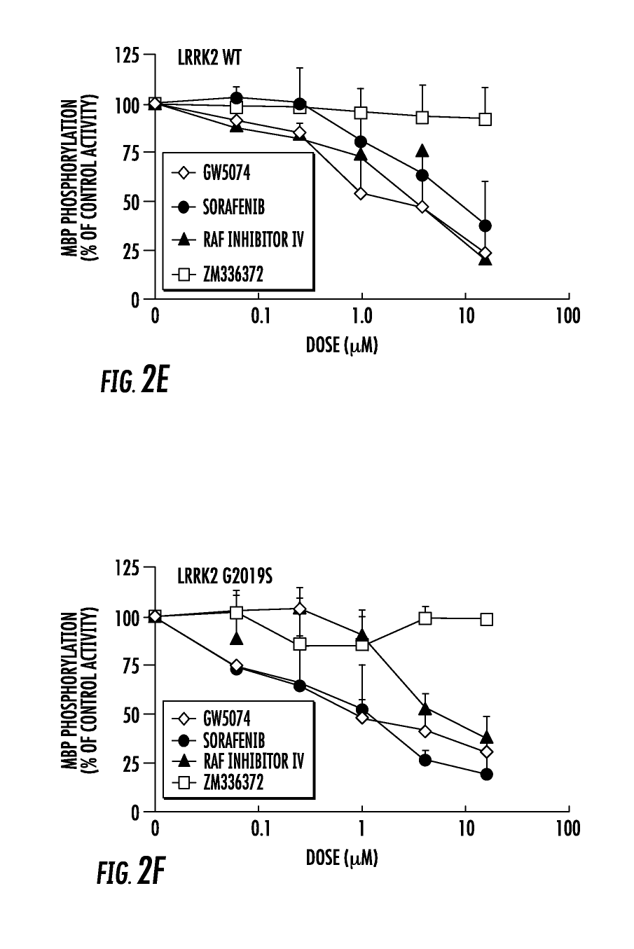

To identify LRRK2 inhibitors, LRRK2 autophosphorylation (FIG. 1) and LRRK2-mediated phosphorylation of myelin basic protein (MBP) were monitored (FIG. 2) in the presence or absence of 84 kinase and phosphatase inhibitors at 16 μM (all from Biomol; see Table 1). Indolinone compounds including staurosporine (compound 6), GF 109203X (compound 31), Ro 31-8220 (compound 33), 5-iodotubercidin (compound 49), GW5074 (compound 56) and indirubin-3′-monooxime (compound 70) and anthracene compounds SP 600125 (compound 68) and damnacanthal (compound 22) substantially inhibited LRRK2 autophosphorylation (FIGS. 1a, b) and LRRK2-mediated phosphorylation of MBP (FIGS. 2a, b). None of the inhibitors substantially enhanced LRRK2 kinase activity.

Table 1 lists all tested compounds including name and structure of the 70 kinase inhibitors and the 14 phosphatase inhibitors. The inhibitory effects of compounds on phosphorylation of LRRK2 and MBP normalized to phosphorylation of LRRK2 and MBP in the absence of compound are the mean±S.E.M. from three independent experiments. LRRK2 kinase inhibitors are discussed hereinabove. KN-93 is a negative control inhibitor.

TABLE 1
Screening Results of BioMol Kinase and Phosphatinse Inhibitor Library for LRRK2 Kinase Activity

| | | | LRRK2 auto- | MBP |
|---|---|---|---|---|
| Inhibitor | Compund | | phosphorylation | phosphorylation |
| Number | Name | Chemical Structure | Average ± SEM | Average ± SEM |
| 1 | PD-98059 | 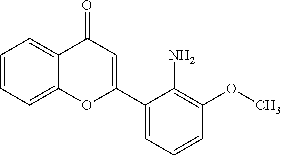 [SMILES: COc1cccc(-c2cc(=O)c3ccccc3o2)c1N] | 77.9 ± 30.6 | 77.5 ± 55.0 |
| 2 | U-0126 | 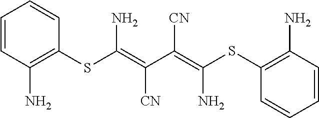 [SMILES: N#CC(=C(/N)Sc1ccccc1N)/C(C#N)=C(\N)Sc1ccccc1N] | 86.9 ± 10.3 | 101.0 ± 105 |
| 3 | SB-203580 | 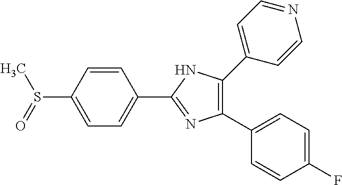 [SMILES: CS(=O)c1ccc(-c2nc(-c3ccc(F)cc3)c(-c3ccncc3)[nH]2)cc1] | 75.1 ± 7.8 | 105.9 ± 15.9 |
| 4 | H-7 | 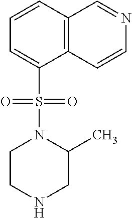 [SMILES: CC1CNCCN1S(=O)(=O)c1cccc2cnccc12] | 80.3 ± 8.2 | 88.0 ± 26.4 |
| 5 | H-9 | 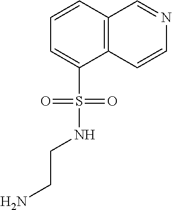 [SMILES: NCCNS(=O)(=O)c1cccc2cnccc12] | 53.8 ± 34.5 | 58.3 ± 22.9 |
| 6 | Staurosporine | 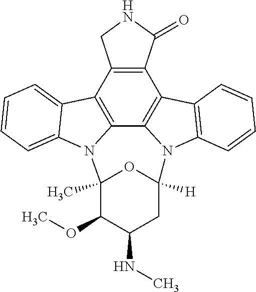 [SMILES: CN[C@@H]1C[C@H]2O[C@@](C)([C@@H]1OC)n1c3ccccc3c3c4c(c5c6ccccc6n2c5c31)C(=O)NC4] | 1.9 ± 1.0 | 9.2 ± 6.4 |
| 7 | AG-494 | 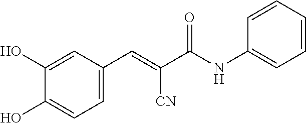 [SMILES: N#C/C(=C\c1ccc(O)c(O)c1)C(=O)Nc1ccccc1] | 56.1 ± 27.6 | 75.6 ± 56.9 |
| 8 | AG-825 | 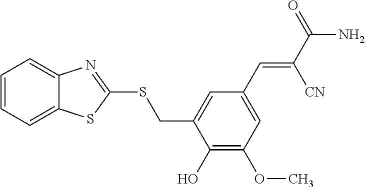 [SMILES: COc1cc(/C=C(\C#N)C(N)=O)cc(CSc2nc3ccccc3s2)c1O] | 81.7 ± 27.1 | 81.5 ± 17.6 |
| 9 | Lavendustin A | 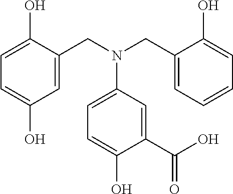 [SMILES: O=C(O)c1cc(N(Cc2ccccc2O)Cc2cc(O)ccc2O)ccc1O] | 84.3 ± 21.1 | 65.2 ± 35.5 |
| 10 | RG-14620 | 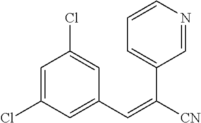 [SMILES: N#C/C(=C/c1cc(Cl)cc(Cl)c1)c1cccnc1] | 90.8 ± 30.7 | 45.2 ± 46.1 |
| 11 | Tyrphostin 23 | 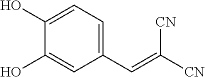 [SMILES: N#CC(C#N)=Cc1ccc(O)c(O)c1] | 66.9 ± 2.0 | 55.5 ± 56.5 |
| 12 | Tyrphostin 25 | 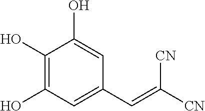 [SMILES: N#CC(C#N)=Cc1cc(O)c(O)c(O)c1] | 62.8 ± 15.8 | 65.9 ± 31.4 |
| 13 | Tyrphostin 46 | 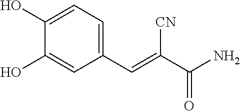 [SMILES: N#C/C(=C\c1ccc(O)c(O)c1)C(N)=O] | 81.0 ± 13.4 | 117.3 ± 18.5 |
| 14 | Tryphostin 47 | 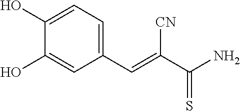 [SMILES: N#C/C(=C\c1ccc(O)c(O)c1)C(N)=S] | 80.5 ± 32.2 | 110.5 ± 29.3 |
| 15 | Tyrphostin 51 | 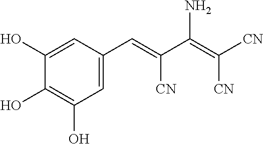 [SMILES: N#CC(C#N)=C(N)/C(C#N)=C/c1cc(O)c(O)c(O)c1] | 79 ± 49.6 | 105.1 ± 19.1 |
| 16 | Tyrphostin 1 | 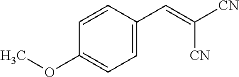 | 111 ± 19.4 | 1746 ± 4.6 |
| 17 | Tyrphostin AG1288 | 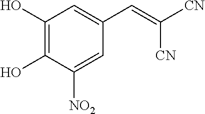 [SMILES: N#CC(C#N)=Cc1cc(O)c(O)c([N+](=O)[O-])c1] | 89 ± 54.9 | 116.0 ± 9.8 |
| 18 | Tyrphostin AG1478 | 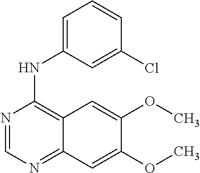 [SMILES: COc1cc2ncnc(Nc3cccc(Cl)c3)c2cc1OC] | 63.7 ± 28.0 | 110.0 ± 44.0 |
| 19 | Tyrphostin AG1295 | 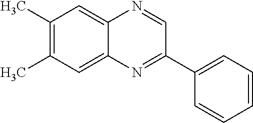 [SMILES: Cc1cc2ncc(-c3ccccc3)nc2cc1C] | 89.4 ± 12.5 | 104.3 ± 14.6 |
| 20 | Tyrphostin 9 | 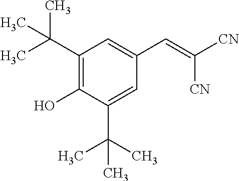 [SMILES: CC(C)(C)c1cc(C=C(C#N)C#N)cc(C(C)(C)C)c1O] | 109.0 ± 19.7 | 120.7 ± 29.4 |
| 21 | Hydroxy-2- naphthalene ethylphosphoric acid | 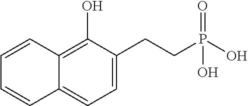 [SMILES: O=P(O)(O)CCc1ccc2ccccc2c1O] | 120.8 ± 54.1 | 138.2 ± 36.9 |
| 22 | Damnacanthal | 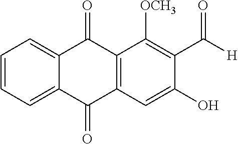 [SMILES: COc1c(C=O)c(O)cc2c1C(=O)c1ccccc1C2=O] | 34.7 ± 6.2 | 35.7 ± 10.3 |
| 23 | Piceatannol | 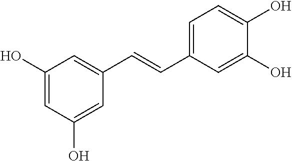 [SMILES: Oc1cc(O)cc(/C=C/c2ccc(O)c(O)c2)c1] | 87 ± 38.6 | 93.0 ± 17.8 |
| 24 | PP1 | 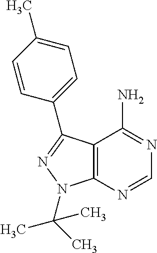 [SMILES: Cc1ccc(-c2nn(C(C)(C)C)c3ncnc(N)c23)cc1] | 88.3 ± 17.1 | 103.6 ± 60.1 |
| 25 | AG-490 | 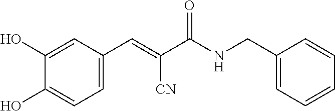 [SMILES: N#C/C(=C\c1ccc(O)c(O)c1)C(=O)NCc1ccccc1] | 78.5 ± 5.8 | 82.9 ± 17.7 |
| 26 | AG-126 | 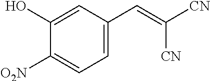 [SMILES: N#CC(C#N)=Cc1ccc([N+](=O)[O-])c(O)c1] | 82.3 ± 10.5 | 114.3 ± 14.1 |
| 27 | AG-370 | 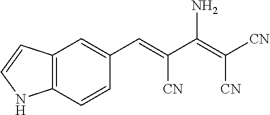 [SMILES: N#CC(C#N)=C(N)/C(C#N)=C/c1ccc2[nH]ccc2c1] | 73.2 ± 8.8 | 79.5 ± 17.8 |
| 28 | AG-879 | 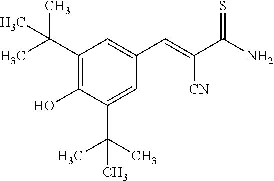 [SMILES: CC(C)(C)c1cc(/C=C(\C#N)C(N)=S)cc(C(C)(C)C)c1O] | 69.8 ± 12.0 | 76.7 ± 4.5 |
| 29 | LY294002 | 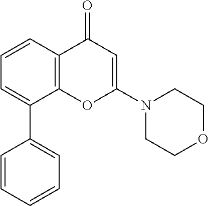 [SMILES: O=c1cc(N2CCOCC2)oc2c(-c3ccccc3)cccc12] | 79.3 ± 3.6 | 75.5 ± 3.1 |
| 30 | Wortmannin | 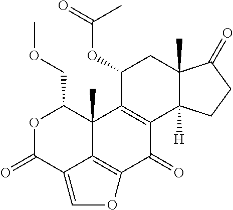 [SMILES: COC[C@H]1OC(=O)c2coc3c2[C@@]1(C)C1=C(C3=O)[C@@H]2CCC(=O)[C@@]2(C)C[C@H]1OC(C)=O] | 83.0 ± 23.6 | 66.8 ± 29.1 |
| 31 | GP109203X | 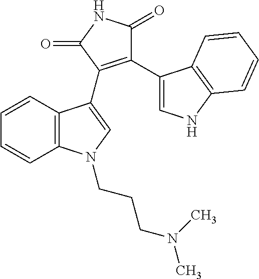 [SMILES: CN(C)CCCn1cc(C2=C(c3c[nH]c4ccccc34)C(=O)NC2=O)c2ccccc21] | 16.1 ± 8.8 | 13.0 ± 8.2 |
| 32 | Hypercin | 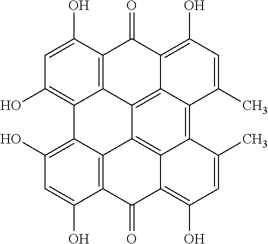 [SMILES: Cc1cc(O)c2c(=O)c3c(O)cc(O)c4c5c(O)cc(O)c6c(=O)c7c(O)cc(C)c8c1c2c(c34)c(c78)c65] | 57.4 ± 30.1 | 160.9 ± 12 |
| 33 | Ro31-8220 | 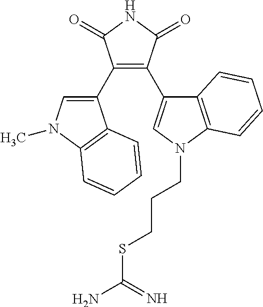 [SMILES: Cn1cc(C2=C(c3cn(CCCSC(=N)N)c4ccccc34)C(=O)NC2=O)c2ccccc21] | 5.4 ± 3.0 | 26.0 ± 38.5 |
| 34 | Sphingosine | 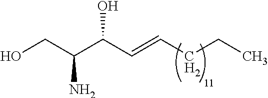 | 80.1 ± 12.8 | 140.0 ± 16.5 |
| 35 | H-89 | 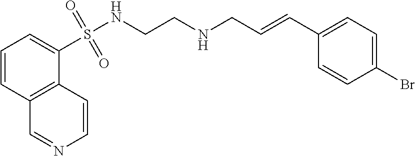 [SMILES: O=S(=O)(NCCNC/C=C/c1ccc(Br)cc1)c1cccc2cnccc12] | 90.8 ± 2.5 | 85.3 ± 54.2 |
| 36 | H-8 | 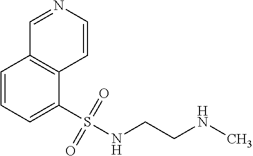 [SMILES: CNCCNS(=O)(=O)c1cccc2cnccc12] | 80.9 ± 3.0 | 50.2 ± 29.4 |
| 37 | HA-1004 | 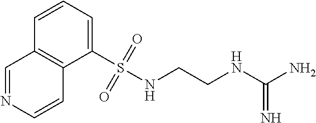 [SMILES: N=C(N)NCCNS(=O)(=O)c1cccc2cnccc12] | 81.1 ± 17.3 | 106.7 ± 25.4 |
| 38 | HA-1077 | 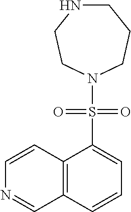 [SMILES: O=S(=O)(c1cccc2cnccc12)N1CCCNCC1] | 84.3 ± 21.9 | 120.4 ± 64.7 |
| 39 | 2-hydroxy-5-(2,5- dihydroxybenzyl- amino) benzoic acid | 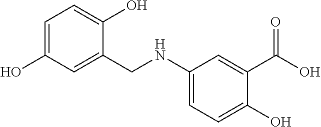 [SMILES: O=C(O)c1cc(NCc2cc(O)ccc2O)ccc1O] | 103.0 ± 4.4 | 148.9 ± 71.4 |
| 40 | KN-62 | 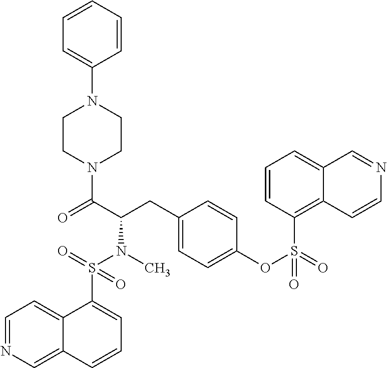 [SMILES: CN([C@@H](Cc1ccc(OS(=O)(=O)c2cccc3cnccc23)cc1)C(=O)N1CCN(c2ccccc2)CC1)S(=O)(=O)c1cccc2cnccc12] | 83.1 ± 22.1 | 125.2 ± 68.8 |
| 41 | KN-93 | 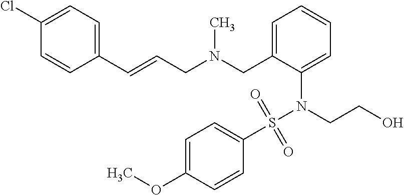 [SMILES: COc1ccc(S(=O)(=O)N(CCO)c2ccccc2CN(C)C/C=C/c2ccc(Cl)cc2)cc1] | 104.6 ± 26.6 | 164.7 ± 40.0 |
| 42 | ML-7 | 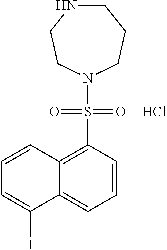 [SMILES: O=S(=O)(c1cccc2c(I)cccc12)N1CCCNCC1 ; Cl] | 87.3 ± 14.8 | 89.4 ± 71.9 |
| 43 | ML-9 | 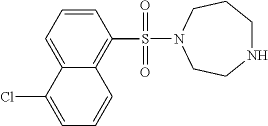 [SMILES: O=S(=O)(c1cccc2c(Cl)cccc12)N1CCCNCC1] | 71.6 ± 2.1 | 120.2 ± 39.6 |
| 44 | 2-aminopurine | 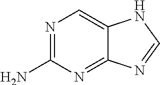 | 95.3 ± 5.9 | 77.2 ± 86.4 |
| 45 | N9-isopropyl olomoucine | 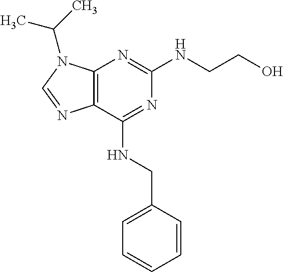 [SMILES: CC(C)n1cnc2c(NCc3ccccc3)nc(NCCO)nc21] | 56.2 ± 30.8 | 111.7 ± 69.7 |
| 46 | Olomoucine | 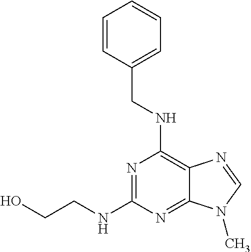 [SMILES: Cn1cnc2c(NCc3ccccc3)nc(NCCO)nc21] | 72.5 ± 2.1 | 125.3 ± 45.6 |
| 47 | Iso-olomoucine | 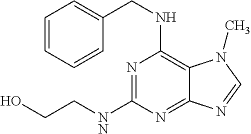 | 92.1 ± 22.2 | 140.7 ± 66.5 |
| 48 | Roscovitine | 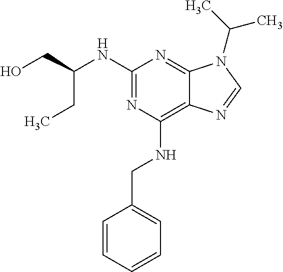 [SMILES: CC[C@@H](CO)Nc1nc(NCc2ccccc2)c2ncn(C(C)C)c2n1] | 69.4 ± 30.1 | 100.1 ± 41.5 |
| 49 | 5-iodo-tubercidin | 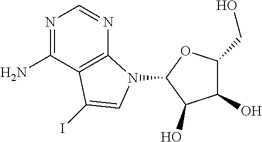 [SMILES: Nc1ncnc2c1c(I)cn2[C@@H]1O[C@H](CO)[C@@H](O)[C@H]1O] | 29.4 ± 14.7 | 39.3 ± 9.9 |
| 50 | LFM-A13 | 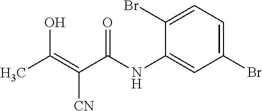 [SMILES: C/C(O)=C(\C#N)C(=O)Nc1cc(Br)ccc1Br] | 66.8 ± 14.2 | 138.8 ± 53.5 |
| 51 | SB-202190 | 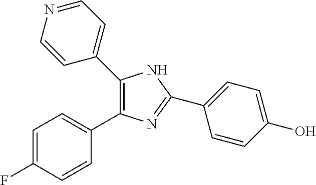 [SMILES: Oc1ccc(-c2nc(-c3ccc(F)cc3)c(-c3ccncc3)[nH]2)cc1] | 64.9 ± 20.9 | 125.5 ± 52.6 |
| 52 | PP2 | 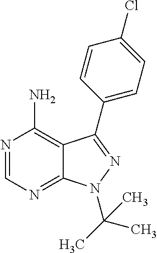 [SMILES: CC(C)(C)n1nc(-c2ccc(Cl)cc2)c2c(N)ncnc21] | 45.7 ± 22.1 | 98.1 ± 44.2 |
| 53 | ZM336372 | 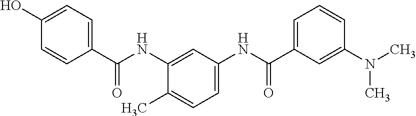 [SMILES: Cc1ccc(NC(=O)c2cccc(N(C)C)c2)cc1NC(=O)c1ccc(O)cc1] | 89.2 ± 2.7 | 152.6 ± 38.0 |
| 54 | SU4312 | 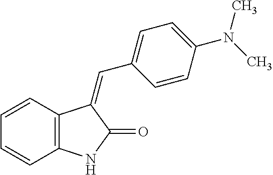 [SMILES: CN(C)c1ccc(/C=C2\C(=O)Nc3ccccc32)cc1] | 37.5 ± 13.9 | 112.8 ± 34.9 |
| 55 | AG-1296 | 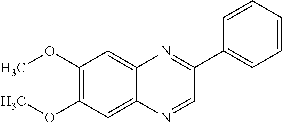 [SMILES: COc1cc2ncc(-c3ccccc3)nc2cc1OC] | 61.2 ± 9.6 | 131.7 ± 34.2 |
| 56 | GW5074 | 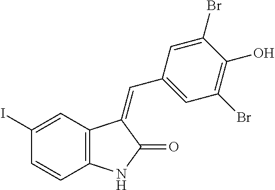 [SMILES: O=C1Nc2ccc(I)cc2/C1=C/c1cc(Br)c(O)c(Br)c1] | 4.6 ± 2.9 | 4.7 ± 4.1 |
| 57 | Palmitoyl-DL- carnitine Cl | 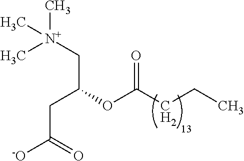 | 78.0 ± 15.4 | 145.3 ± 85.0 |
| 58 | Rottlerin | 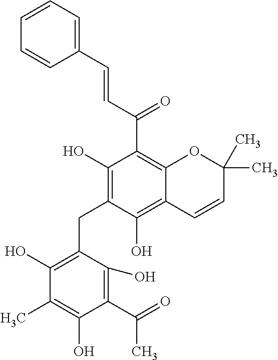 [SMILES: CC(=O)c1c(O)c(C)c(O)c(Cc2c(O)c3c(c(C(=O)/C=C/c4ccccc4)c2O)OC(C)(C)C=C3)c1O] | 28.1 ± 39.8 | 147.2 ± 64.3 |
| 59 | Genistein | 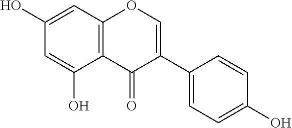 [SMILES: O=c1c(-c2ccc(O)cc2)coc2cc(O)cc(O)c12] | 69.4 ± 37.9 | 63.1 ± 66.7 |
| 60 | Daidzein | 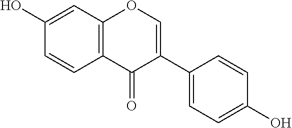 [SMILES: O=c1c(-c2ccc(O)cc2)coc2cc(O)ccc12] | 81.0 ± 31.9 | 141.0 ± 50.8 |
| 61 | Erbstatin analog | 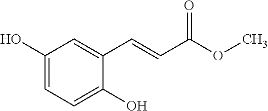 | 73.0 ± 5.3 | 93.9 ± 11.5 |
| 62 | Quercetin dihydrate | 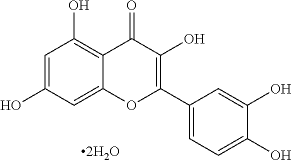 [SMILES: O=c1c(O)c(-c2ccc(O)c(O)c2)oc2cc(O)cc(O)c12] | 39.2 ± 39.9 | 70.1 ± 40.5 |
| 63 | SU1498 | 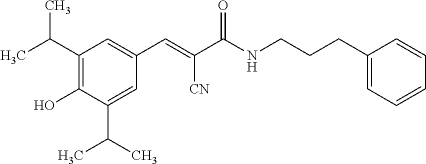 [SMILES: CC(C)c1cc(/C=C(\C#N)C(=O)NCCCc2ccccc2)cc(C(C)C)c1O] | 68.5 ± 1.5 | 101.3 ± 25.3 |
| 64 | ZM449829 | 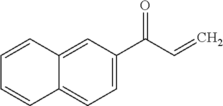 | 70.7 ± 4.2 | 123.7 ± 26.3 |
| 65 | BAY11-7082 | 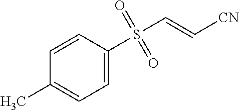 | 75.1 ± 12.8 | 124.8 ± 23.5 |
| 66 | 5,6-dichloro-1- beta-D- ribofutanosyl- benzimidiazole | 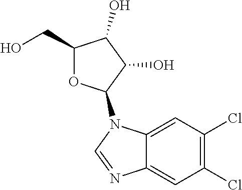 [SMILES: OC[C@@H]1O[C@H](n2cnc3cc(Cl)c(Cl)cc32)[C@@H](O)[C@H]1O] | 56.4 ± 12.9 | 120.8 ± 78.0 |
| 67 | 2,2′,3,3′,4,4′- hexahydroxy- 1,1′-biphenyl- 6,6′-dimenthanol dimethyl ester | 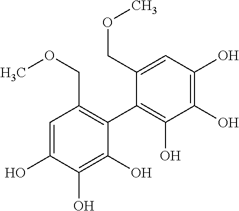 [SMILES: COCc1cc(O)c(O)c(O)c1-c1c(COC)cc(O)c(O)c1O] | 79.2 ± 24.2 | 123.2 ± 33.8 |
| 68 | SP600125 | 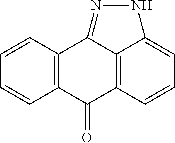 [SMILES: O=C1c2ccccc2-c2n[nH]c3cccc1c23] | 18.3 ± 22.8 | 31.2 ± 12.1 |
| 69 | Indirubin | 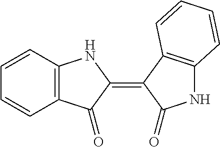 [SMILES: O=C1Nc2ccccc2/C1=C1\Nc2ccccc2C1=O] | 82.0 ± 4.2 | 110.3 ± 28.4 |
| 70 | Indirubin-3- monooxine | 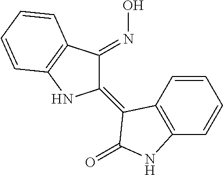 [SMILES: O=C1Nc2ccccc2/C1=C1/Nc2ccccc2/C1=N\O] | 6.0 ± 6.5 | 33.1 ± 15.7 |
| 71 | Cantharidic acid | 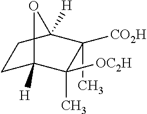 | 75.9 ± 2.1 | 99.8 ± 3.4 |
| 72 | Cantharidin | 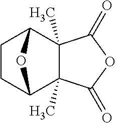 | 69.1 ± 8.8 | 109.7 ± 22.4 |
| 73 | Endothall | 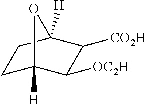 | 78.8 ± 5.1 | 128.9 ± 9.4 |
| 74 | Benzyl- phosphoric acid | 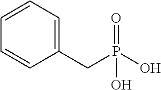 [SMILES: O=P(O)(O)Cc1ccccc1] | 68.3 ± 1.85 | 121.8 ± 23.6 |
| 75 | L-p-bromo- tetraamisole oxalate | 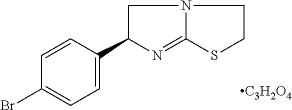 | 75.9 ± 3.9 | 83.2 ± 50.5 |
| 76 | RK-682 | 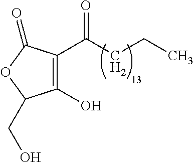 | 56.8 ± 38.9 | 161.6 ± 30.1 |
| 77 | RWJ-60475 | 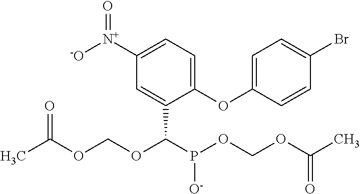 | 52.0 ± 31.4 | 95.7 ± 57.3 |
| 78 | Levarmisole HCl | 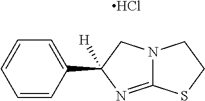 [SMILES: Cl ; c1ccc([C@H]2CN3CCSC3=N2)cc1] | 74.6 ± 1.7 | 133.2 ± 17.6 |
| 79 | Tetramisole HCl | 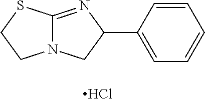 [SMILES: c1ccc(C2CN3CCSC3=N2)cc1 ; Cl] | 75.0 ± 2.3 | 138.8 ± 33.9 |
| 80 | Cypermethrin | 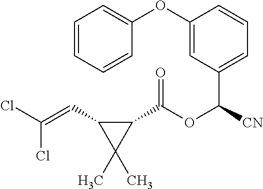 [SMILES: CC1(C)[C@H](C=C(Cl)Cl)[C@@H]1C(=O)O[C@H](C#N)c1cccc(Oc2ccccc2)c1] | 66.4 ± 17.4 | 76.9 ± 13.7 |
| 81 | Deltamethrin | 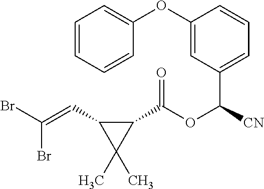 [SMILES: CC1(C)[C@H](C=C(Br)Br)[C@@H]1C(=O)O[C@H](C#N)c1cccc(Oc2ccccc2)c1] | 73.9 ± 2.2 | 99.6 ± 23.1 |
| 82 | Fenvaierate | 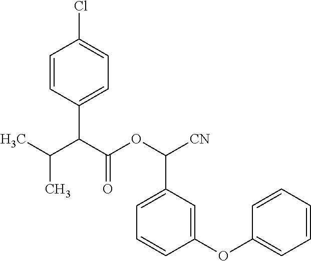 [SMILES: CC(C)C(C(=O)OC(C#N)c1cccc(Oc2ccccc2)c1)c1ccc(Cl)cc1] | 80.4 ± 13.0 | 61.5 ± 54.0 |
| 83 | Tyrphostin 8 | 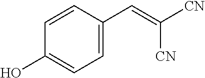 [SMILES: N#CC(C#N)=Cc1ccc(O)cc1] | 68.6 ± 35.5 | 118.2 ± 51.8 |
| 84 | Cinngel | 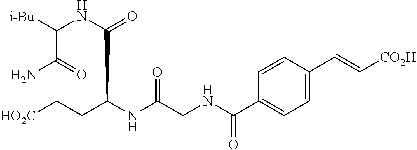 | 63.9 ± 12.0 | 124.1 ± 37.2 |

The half-maximal inhibitory concentrations of these eight inhibitors were determined against autophosphorylation and MBP phosphorylation by wild type (WT) and G2019S LRRK2 (FIGS. 1c, d, FIGS. 2c, d and Table 2). As provided immediately herein below, Table 2 provides IC₅₀ value of kinase inhibitors for LRRK2 WT and LRRK2 G2019S kinase activity. The table lists IC₅₀ value (μM) of eight LRRK2 inhibitors on LRRK2 WT and LRRK2 G2019S kinase activity from the mean of three independent experiments.

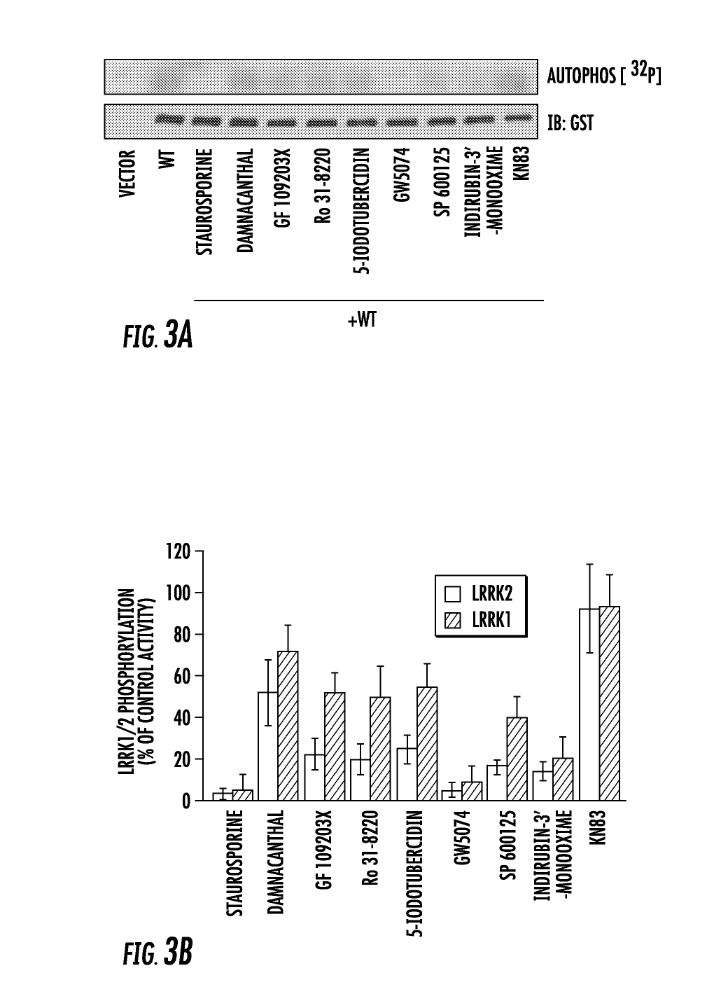

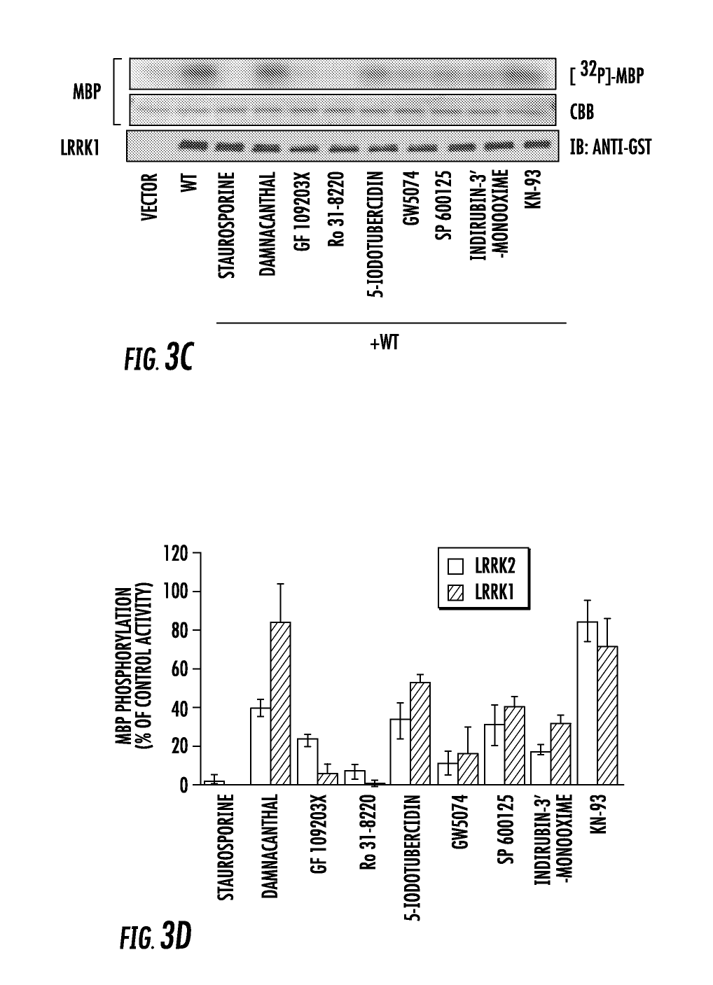

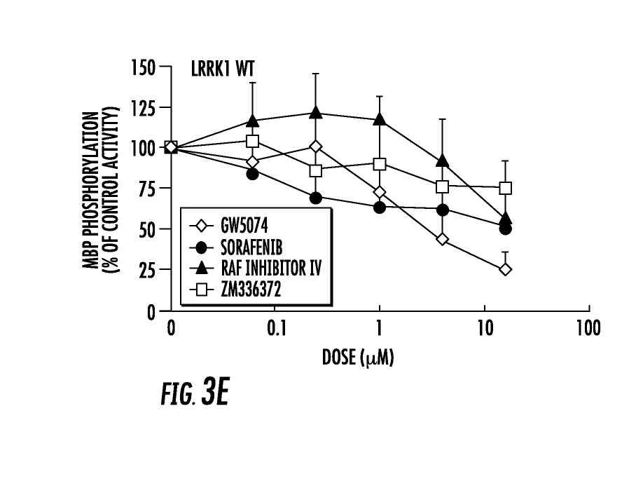

All of the inhibitors except indirubin-3′-monooxime had relatively similar potencies against WT and G2019S LRRK2 autophosphorylation activity (FIGS. 1c, d and Table 2). Indirubin-3′-monooxime more potently inhibited G2019S LRRK2 autophosphorylation (FIGS. 1c, d and Table 2). Staurosporine, damnacanthal, SP 600125, and 5-iodotubercidin equivalently inhibited both WT and G2019S LRRK2 MBP phosphorylation (FIGS. 2c, d and Table 2). Both protein kinase C inhibitors, Ro 31-8220 and GF109203X, more potently inhibited both WT and G2019S LRRK2 MBP phosphorylation, and GW5074 was less potent in inhibiting both WT and G2019S LRRK2 MBP phosphorylation than WT and G2019S LRRK2 autophosphorylation (FIGS. 1c, d, FIGS. 2c, d and Table 2). All eight inhibitors had a similar inhibitory profile against LRRK1 autophosphorylation and MBP phosphorylation (FIG. 3a-d).

TABLE 2
IC₅₀ Value of Kinase Inhibitors for LRRK2 WT and
G2019S Kinase Activity.

| | LRRK2 WT | | LRRK2 G2019S | |
|---|---|---|---|---|
| | IC₅₀(μM) | | IC₅₀(μM) | |
| | LRRK2 | MBP | LRRK2 | MBP |
| | autophos- | phos- | autophos- | phos- |
| | phorylation | phorylation | phorylation | phorylation |
| Staurosporine | 0.04 | 0.04 | 0.04 | 0.04 |
| Damnacanthal | 19.14 | 7.81 | 5.25 | 9.45 |
| GF 109203X | 5.27 | 2.19 | 17.04 | 2.62 |
| Ro 31-8220 | 1.64 | 0.05 | 4.51 | 5.16 |
| 5-lodotuber- | 3.21 | 14.78 | 4.23 | 3.41 |
| cidin | | | | |
| GW 5074 | 0.88 | 3.15 | 0.22 | 0.88 |
| SP 600125 | 3.71 | 3.10 | 3.37 | 5.00 |
| Indirubin-3′- | 1.75 | 4.83 | 0.38 | 1.31 |
| monooxime | | | | |

Given that LRRK2 and LRRK1 are related to the MAP kinase kinase kinase Raf, Mata, I. F., et al., Trends Neurosci. 29, 286-293 (2006), and GW5074 inhibits Raf kinase, Chin, P. C. et al. J. Neurochem. 90, 595-608 (2004), LRRK2 and LRRK1 autophosphorylation and MBP phosphorylation were monitored in the presence or absence of additional Raf kinase inhibitors ZM336372, sorafenib and Raf inhibitor IV (FIG. 1e). GW5074 more potently inhibited G2019S LRRK2 autophosphorylation and MBP phosphorylation than it did WT LRRK1 autophosphorylation and MBP phosphorylation (FIGS. 1f, g, FIGS. 2e, f and 3e and Table 3).

As provided immediately herein below, Table 3 provides IC₅₀ value of kinase inhibitors for LRRK1 WT, LRRK2 WT, and LRRK2 G2019S kinase activity. The table lists IC₅₀ value (μM) of four different Raf kinase inhibitor on LRRK1 WT, LRRK2 WT, and LRRK2 G2019S kinase activity from the mean of three independent experiments.

TABLE 3
IC₅₀ Value of Kinase Inhibitors for LRRK1 and LRRK2 Kinase Artivity.

| | LRRK1 WT | | LRRK2 WT | | LRRK2 G2019S | |
|---|---|---|---|---|---|---|
| | IC₅₀ (μm) | | IC₅₀ (μm) | | IC₅₀ (μm) | |
| | LRRK1 | MBP | LRRK2 | MBP | LRRK2 | MBP |
| | autophosphorylation | phosphorylation | autophosphorylation | phosphorylation | autophosphorylation | phosphorylation |
| GW5074 | 0.73 | 3.25 | 0.88 | 1.09 | 0.22 | 0.88 |
| ZM336372 | NA | NA | NA | NA | NA | NA |
| Sorafenib | NA | 13.82 | 14.56 | 5.58 | 1.17 | 1.23 |
| Raf inhibitor IV | NA | 21.53 | 5.43 | 3.61 | 3.94 | 6.51 |
| Indirubin-3′- | 0.79 | 3.10 | 1.75 | 4.83 | 0.38 | 1.31 |
| monooxime | | | | | | |
| Indirubin | NA | NA | NA | NA | NA | NA |

ZM336372 had minimal to no effect on LRRK1 autophosphorylation and MBP phosphorylation and no effect on WT or G2019S LRRK2 autophosphorylation and MBP phosphorylation (FIG. 1e-g, FIGS. 2e, f and 3e and Table 3). Both sorafenib and Raf inhibitor IV inhibited LRRK2 autophosphorylation and MBP phosphorylation with less potency than GW5074, but they had minimal to no effect on LRRK1 autophosphorylation or MBP phosphorylation (FIG. 1e-g, FIGS. 2e, f and 3e and Table 3). These results indicate that GW5074 inhibits both LRRK2 and LRRK1 kinase activities, whereas sorafenib and Raf inhibitor IV are relatively selective for LRRK2 kinase activity and ZM336372 has minimal to no effect on LRRK2 and LRRK1 kinase activities.

Indirabin-3′-monooxime and the related analog indirubin were compared against WT LRRK1, WT LRRK2 and G2019S LRRK2 autophosphorylation and MBP phosphorylation. Indirubin-3′-monooxime inhibited WT LRRK1, WT LRRK2 and G2019S LRRK2 autophosphorylation and MBP phosphorylation, whereas indirubin had no effect on WT LRRK1, WT LRRK2 and G2019S LRRK2 autophosphorylation or MBP phosphorylation (Table 3). GW5074 and indirubin-3′-monooxime also inhibited LRRK2-mediated phosphorylation of eukaryotic translation initiation factor 4E-binding protein (4E-BP1), a putative physiologic LRRK2 substrate, Imai, Y. et al. EMBO J. 27, 2432-2443 (2008), whereas ZM336372 and indirubin did not inhibit LRRK2 phosphorylation of 4E-BP1 (FIGS. 1h, i).

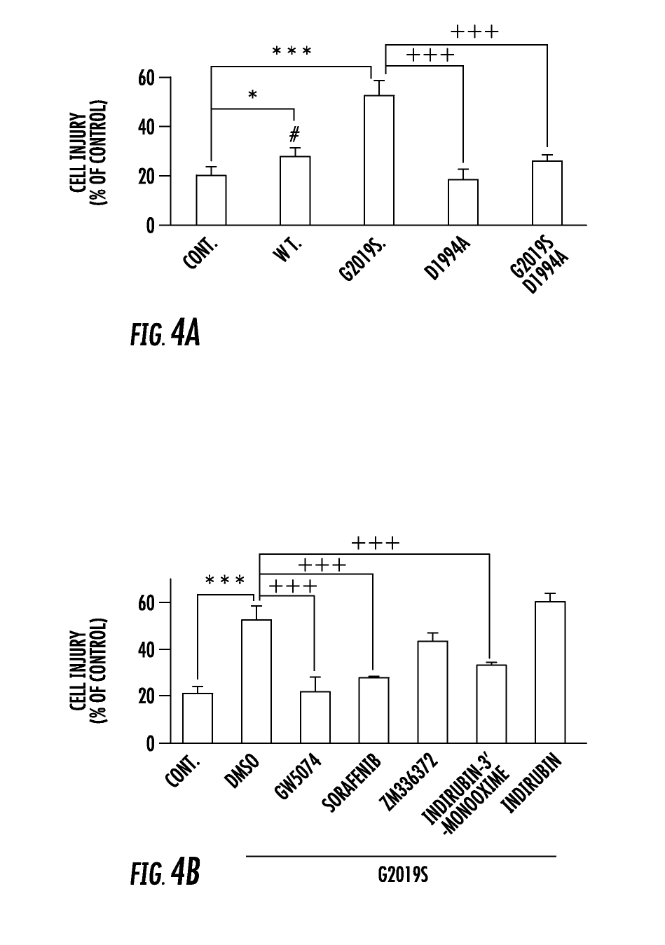

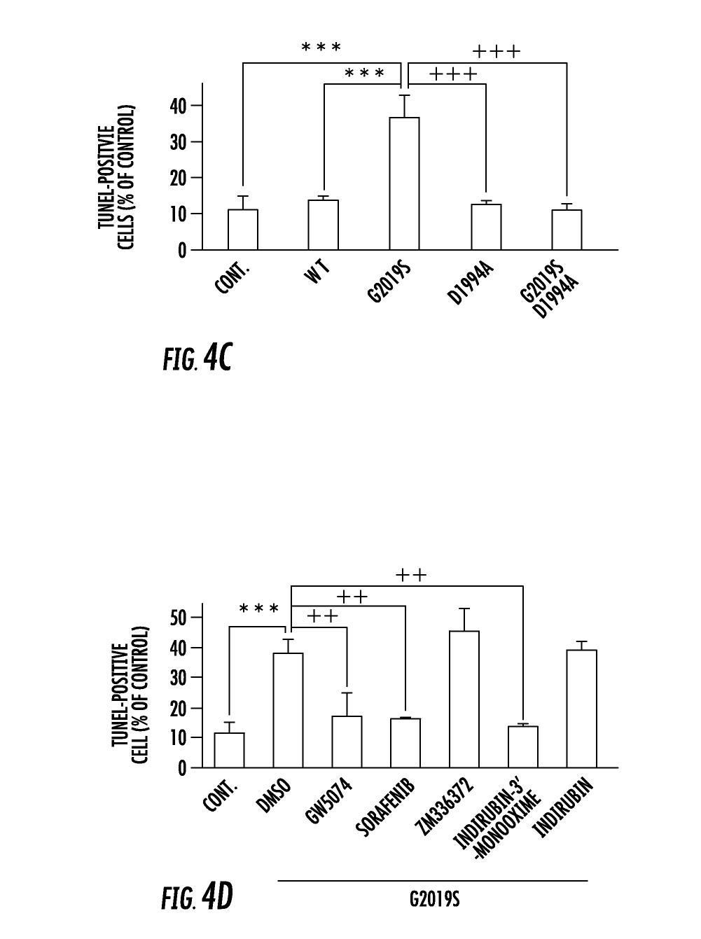

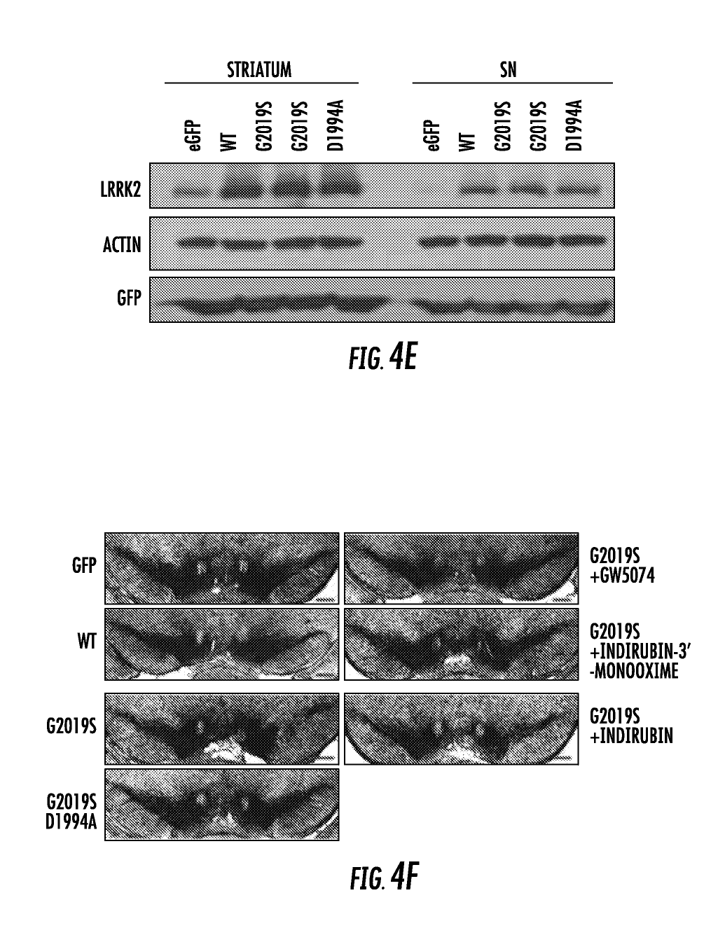

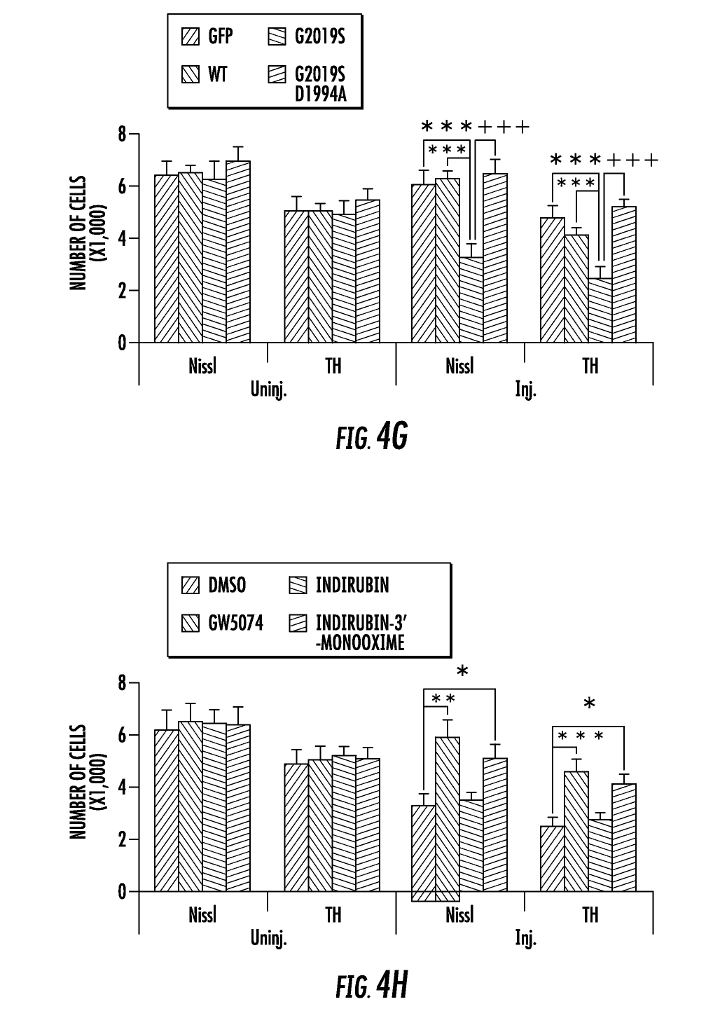

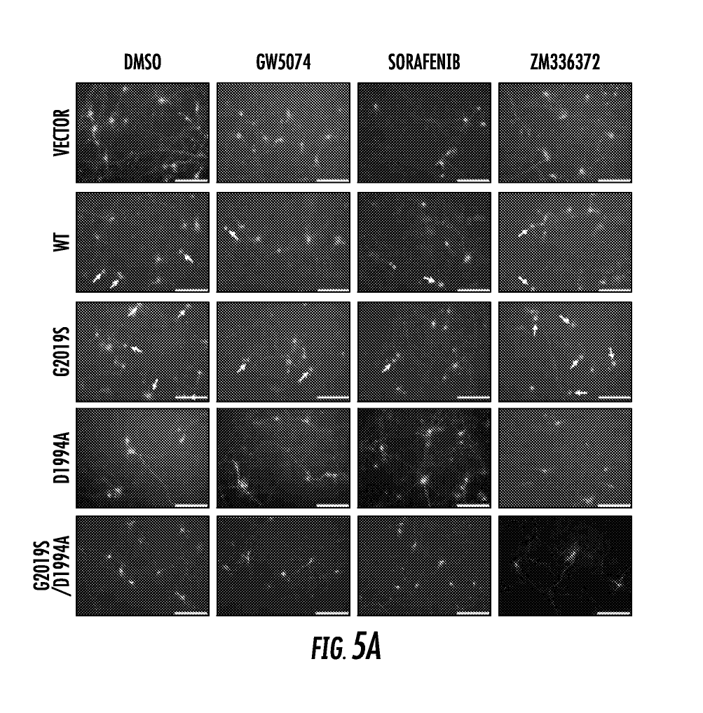

Both WT LRRK2 and G2019S LRRK2 overexpression led to primary cortical neuron injury, as assessed by neurite shortening (FIGS. 4a, b and FIG. 5), and G2019S LRRK2 overexpression led to cell death, as assessed by DNA fragmentation. (TUNEL assay) (FIGS. 4c, d and Methods), whereas kinase-dead (D1994A) versions of LRRK2 and G2019S LRRK2 were devoid of toxicity, as previously described (FIG. 4a-d and FIG. 5). Smith, W. W. et al. Nat. Neurosci. 9, 1231-1233 (2006); West, A. B. et al. Hum. Mol. Genet. 16, 223-232 (2007); Smith, W. W. et al. Proc. Natl. Acad. Sci. USA 102, 18676-18681 (2005). Treatment of the cortical cultures with 0.5 μM GW5074 and 0.5 μM indirubin-3′-monooxime, which inhibit both LRRK2 and LRRK1, attenuated G2019S LRRK2 cell injury and cell death (FIGS. 4b, d). The Raf kinase inhibitor sorafenib (0.5 μM), which is relatively selective for LRRK2 (see Table 3), also completely protected against G2019S LRRK2 toxicity (FIGS. 4b, d and FIG. 5). The Raf kinase inhibitor ZM336372 (0.5 μM), which does not inhibit LRRK2 or LRRK1 kinase activity, failed to inhibit G2019S LRRK2 toxicity (FIGS. 4b, d and FIG. 5). The cyclin-dependent kinase and glycogen synthase kinase-3β (GSK-3β) inhibitor indirubin, which does not inhibit LRRK2 or LRRK1 kinase activity, failed to inhibit G2019S LRRK2 toxicity (FIGS. 4b, d and FIG. 5). These results taken together indicate that the protection afforded by the Raf inhibitors (GW5074 and sorafenib) and the cyclin-dependent kinase and GSK3β inhibitor (indirubin-3-monooxime) are due to inhibition of LRRK2 kinase activity and not inhibition of Raf, cyclin-dependent or GSK-β kinase activity.

To determine the efficacy of the LRRK2 kinase inhibitors in vivo, a herpes simplex virus (HSV) amplicon-based mouse model of LRRK2 dopaminergic neurotoxicity was developed (FIG. 4e-h and Methods). GFP was extensively co-expressed with tyrosine hydroxylase and in ˜75% of substantia nigra compacta neurons after an intrastriatal HSV-eGFP injection (FIG. 6). Immunoblot analysis confirmed that WT, G2019S and G2019S-D1994A LRRK2 are overexpressed in equivalent amounts (FIG. 4e). HSV amplicon-mediaied delivery of LRRK2 G2019S induced significant loss of tyrosine hydroxylase-positive neurons 3 weeks after stereotaxic injection into the ipsilateral striatum of mice compared to control viruses expressing WT LRRK2 and eGFP (FIGS. 4f, g). HSV amplicon-mediated delivery of G2019S-D1994A LRRK2 caused no neuronal loss, similarly to WT LRRK2 and GFP control viruses (FIGS. 4f, g).

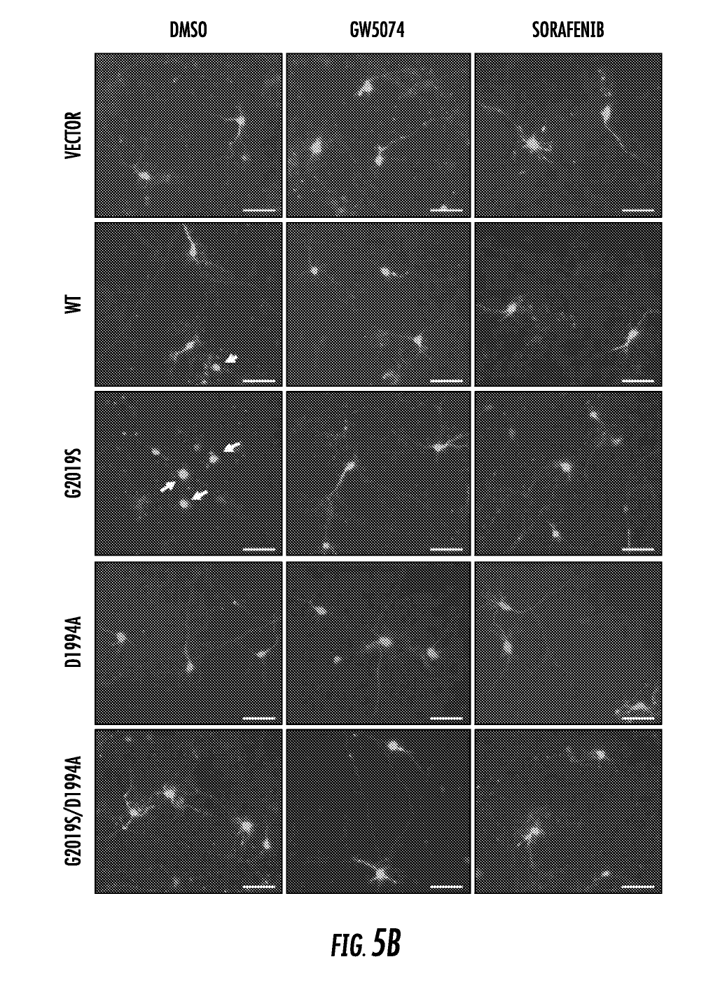

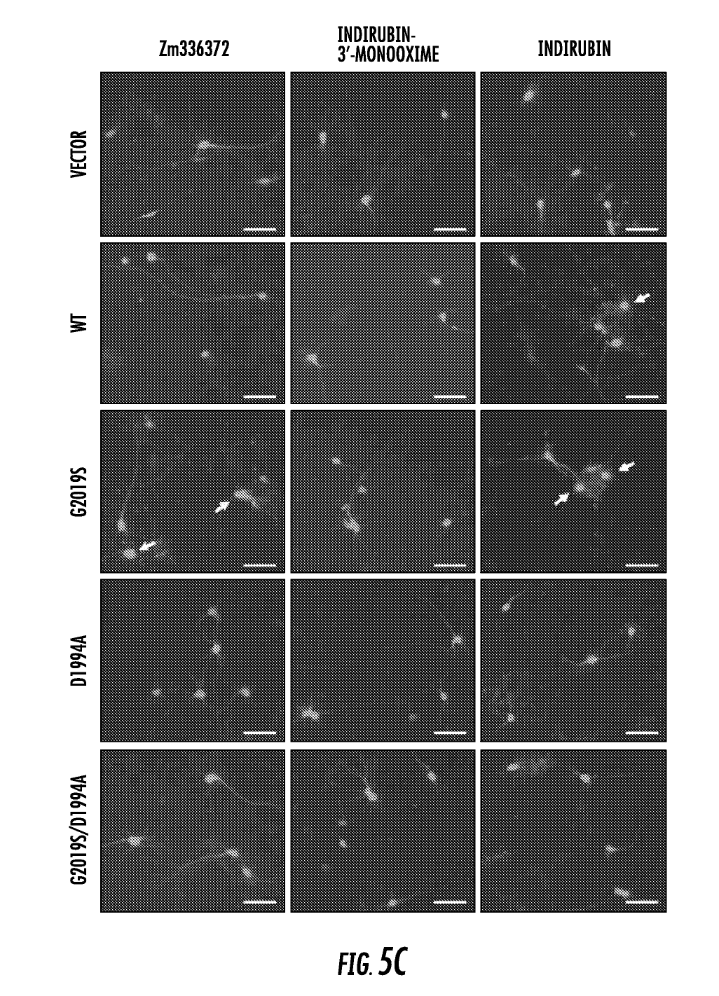

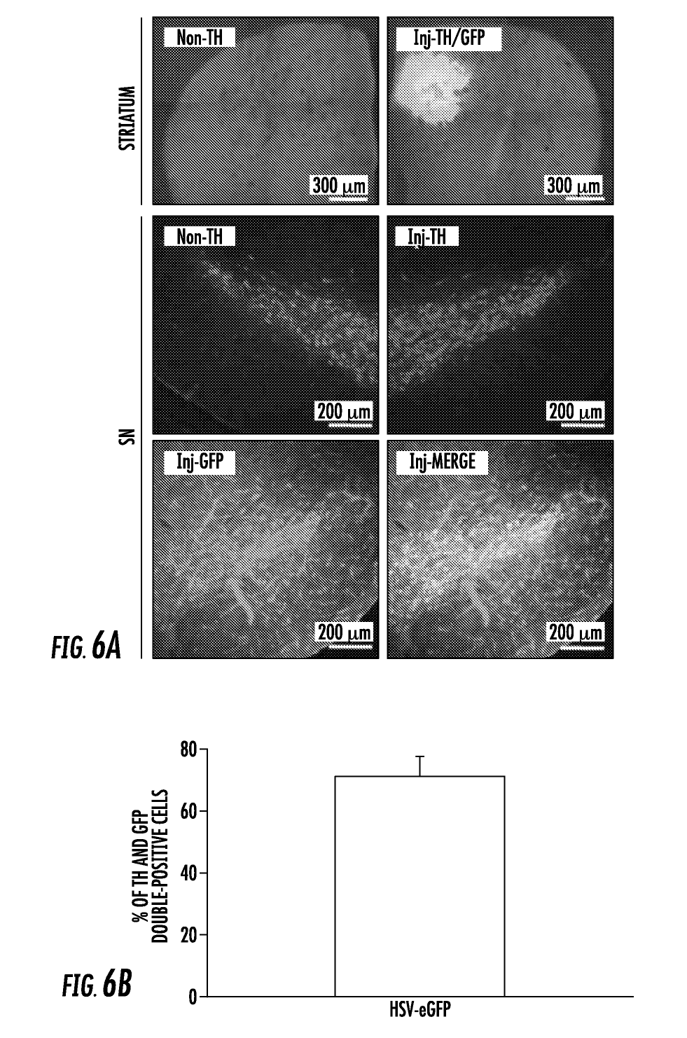

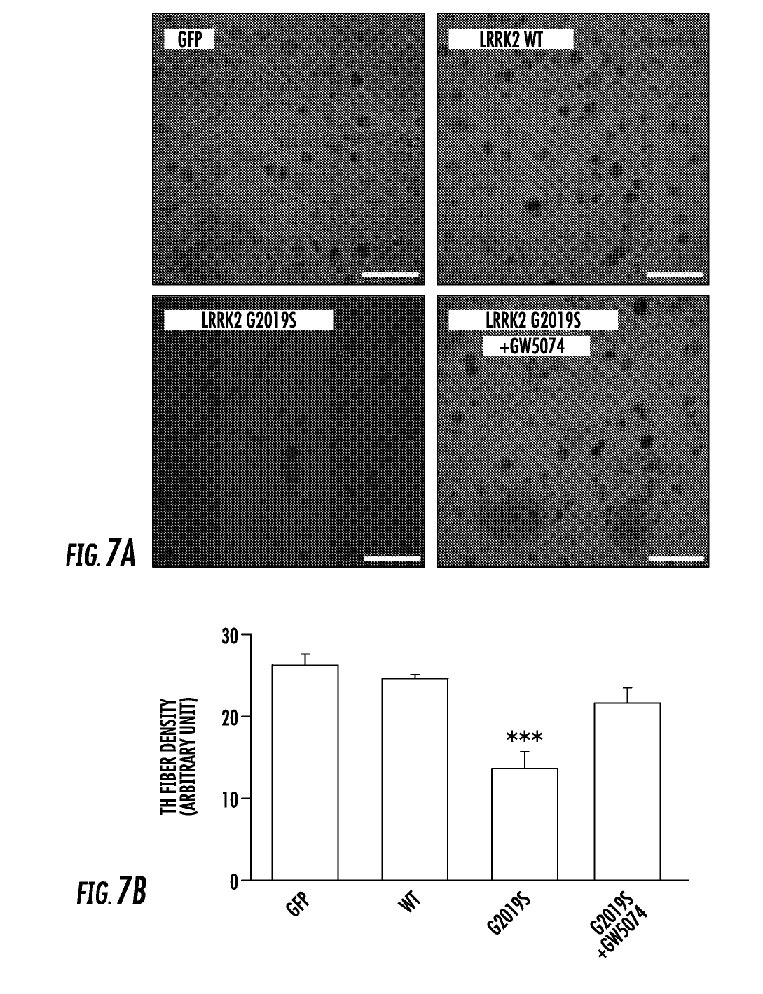

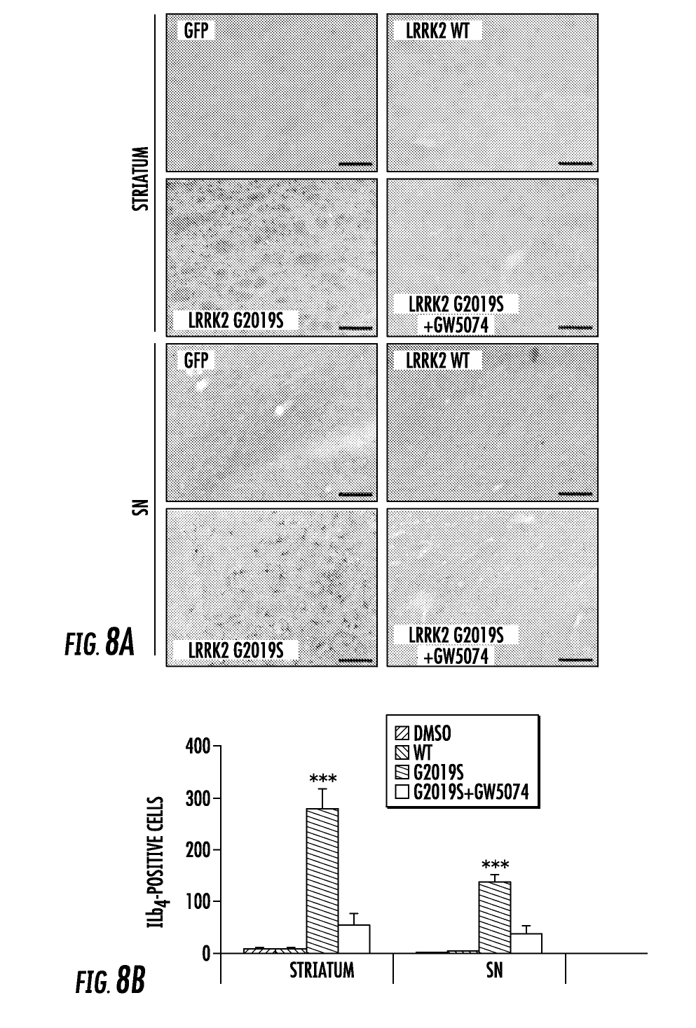

Because GW5074 and indirubin-3′-monooxime and indirubin are known to cross the blood-brain barrier, Chin, P. C. et al. J. Neurochem. 90, 595-608 (2004); Leclerc, S. et al. J. Biol. Chem. 276, 251-260 (2001); Wang, W. et al. Neuropharmacology 52, 1678-1684 (2007), they were selected to test whether inhibition of LRRK2 kinase activity is protective in vivo. Twice daily injections of the LRRK2 kinase inhibitors, GW5074 and indirubin-3′-monooxime, (2.5 mg per kg body weight intraperitoneally) attenuated the loss of tyrosine hydroxylase-positive neurons induced by HSV-G2019S LRRK2 compared to DMSO- and indirubin-injected controls (FIGS. 4f, h). The density of tyrosine hydroxylase-positive fibers also was reduced in mice given HSV-LRRK2 G2019S compared to those given HSV-eGFP control and HSV-WT LRRK2, and the reduction in the density of tyrosine hydroxylase-positive fibers was rescued by GW5074 (FIG. 7). Mice transduced with eGFP and WT LRRK2 did not show any signs of inflammation as determined by isolectin B₄ (ILB4) staining, but G2019S LRRK2 induced a significant increase in ILB4-positive cells in the striatum and substantia nigra pars compacts, which also was prevented by administration of GW5074 (FIG. 8).

In summary, the presently disclosed subject matter identifies kinase inhibitors that inhibit LRRK2 kinase activity and protect against LRRK2 toxicity both in vitro and in vivo. Other kinase inhibitors have recently been reported to inhibit LRRK2 kinase activity with similar potencies to those described here. Anand, V. S. et al. FEBS J. 276, 466-478 (2009); Covy, J. P. & Giasson, B. I. Biochem. Biophys. Res. Commun. 378, 473-477 (2009); Nichols, R. J. et al. Biochem. J. 424, 47-60 (2009); Reichling, L. J. & Riddle, S. M. Biochem. Biophys. Res. Commun. 384, 255-258 (2009). Further, the presently disclosed subject matter demonstrates that pharmacologic inhibition of LRRK2 kinase activity is a potentially promising therapeutic modality for treating neurodegeneration in Parkinson's disease.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Gasser, T. Expert Rev. Mol. Med. 11, e22 (2009).
Greggio, E. et al. Neurobiol. Dis. 23, 329-341 (2006).
Smith, W. W. et al. Nat. Neurosci. 9, 1231-1233 (2006).
West, A. B. et al. Hum. Mol. Genet. 16, 223-232 (2007).
Whaley, N. R., Uitti, R. J., Dickson, D. W., Farrer, M. J. & Wszolek, Z. K. J. Neural Transm. Suppl. 70, 221-229 (2006).
Mata, I. F., Wedemeyer, W. J., Farrer, M. J., Taylor, J. P. & Gallo, K. A. Trends Neurosci. 29, 286-293 (2006).
Chin, P. C. et al. J. Neurochem. 90, 595-608 (2004).
Imai, Y. et al. EMBO J. 27, 2432-2443 (2008).
Smith, W. W. et al. Proc. Natl. Acad. Sci. USA 102, 18676-18681 (2005).
Leclerc, S. et al. J. Biol. Chem. 276, 251-260 (2001).
Wang, W. et al. Neuropharmacology 52, 1678-1684 (2007).
Anand, V. S. et al. FEBS J. 276, 466-478 (2009).
Covy, J. P. & Giasson, B. I. Biochem. Biophys. Res. Commun. 378, 473-477 (2009).
Nichols, R. J. et al. Biochem. J. 424, 47-60 (2009).
Reichling, L. J. and Riddle, S. M. Biochem. Biophys. Res. Commun. 384, 255-258 (2009).
West, A. B., et al. Parkinson's disease-associated mutations in leucine-rich repeat kinase 2 augment kinase activity. Proc Natl Acad Sci USA 102, 16842-16847 (2005).
Gonzalez-Zulueta, M., et al. Manganese superoxide dismutase protects nNOS neurons from NMDA and nitric oxide-mediated neurotoxicity. J. Neurosci 18, 2040-2055 (1998).
Smith, W. W., et al. Kinase activity of mutant LRRK2 mediates neuronal toxicity. Nat Neurosci 9, 1231-1233 (2006).
West, A. B., et al. Parkinson's disease-associated mutations in LRRK2 link enhanced GTP-binding and kinase activities to neuronal toxicity. Hum Mol Genet 16, 223-232 (2007).
Maguire-Zeiss, K. A., Bowers, W. J. and Federoff, H. J. HSV vector-mediated gene delivery to the central nervous system. Curr Opin Mol Ther 3, 482-490 (2001).
Bowers, W. J., Howard, D. P., Brooks, A. I., Halterman, M. W. and Federoff, H. J. Expression of vhs and VP16 during HSV-1 helper virus-free amplicon packaging enhances titers. Gene Ther 8, 111-120 (2001).
Bowers, W. J., Howard, D. F. and Federoff, H. J. Discordance between expression and genome transfer titering of HSV amplicon vectors: recommendation for standardized enumeration. Mol Ther 1, 294-299 (2000).
Jin, B. K., et al. Prolonged in vivo gene expression driven by a tyrosine hydroxylase promoter in a defective herpes simplex virus amplicon vector. Hum Gene Ther 7, 2015-2024 (1996).
Gardet, A., et al., “LRRK2 Is Involved in the IFN-γ Response and Host Response to Pathogens,” J. Immunol. 185:5577-5585 (2010).

Although the foregoing subject matter has been described in some detail by way of illustration and example for purposes of clarity of understanding, it will be understood by those skilled in the art that certain changes and modifications can be practiced within the scope of the appended claims.

1. A method for inhibiting a leucine-rich repeat kinase-2 (LRRK2) kinase, the method comprising contacting an LRRK2 kinase with a compound of formulae (I-VII): 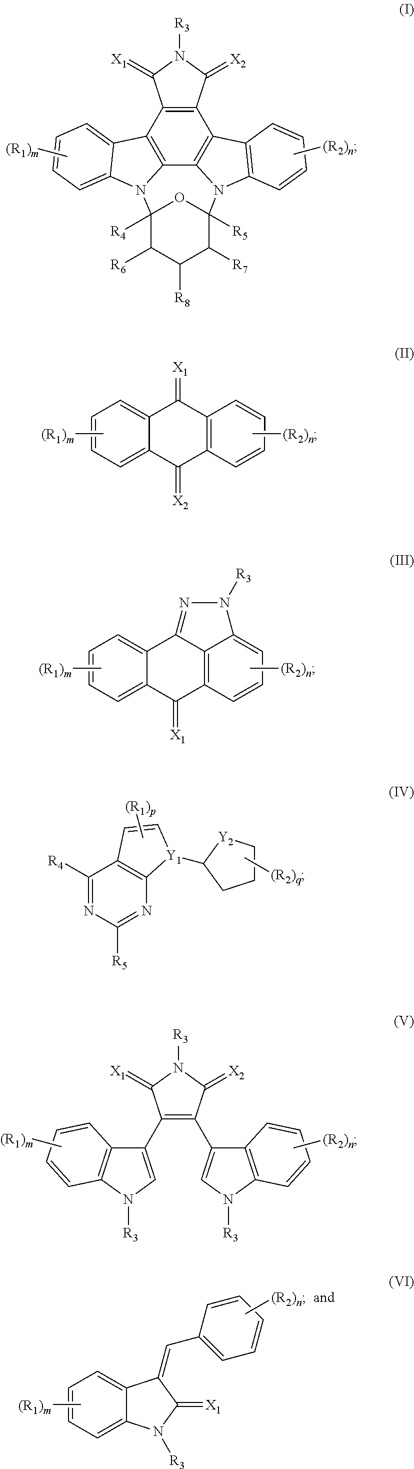 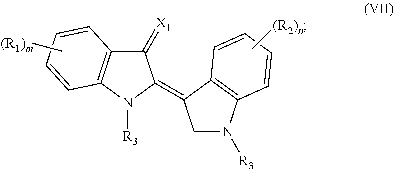
wherein:
p is an integer selected from the group consisting of 0, 1, and 2;
q is an integer selected from the group consisting of 0, 1, 2, and 3;
m and n are each an integer independently selected from the group consisting of 0, 1, 2, 3, and 4;
each (=X₁) and (=X₂) can be present or absent and, when present, each X₁ and X₂ is independently selected from the group consisting of O, S, CR₉R₁₀, and NR₁₁,
wherein R₉, R₁₀, and R₁₁ are each independently selected from the group consisting of H, substituted or unsubstituted alkyl, substituted or substituted heteroalkyl, substituted or substituted alkenyl, alkynyl, substituted or unsubstituted cycloalkyl, substituted or unsubstituted cycloheteroalkyl, substituted or unsubstituted aryl, substituted or unsubstituted heteroaryl, aralkyl, hydroxyl, —COR₁₂, —COOR₁₃, —OR₁₄, wherein R₁₂, R₁₃, and R₁₄ are each independently selected from the group consisting of H, substituted or unsubstituted alkyl, substituted or substituted heteroalkyl, substituted or substituted alkenyl, alkynyl, substituted or unsubstituted cycloalkyl, substituted or unsubstituted cycloheteroalkyl, substituted or unsubstituted aryl, substituted or unsubstituted heteroaryl;
Y₁ is selected from the group consisting of N, and CR₉, wherein R₉ is as defined above;
Y₂ is selected from the group consisting of O, S, CR₉R₁₀, and NR₁₁, wherein R₉, R₁₀, and R₁₁ are as defined above;
each R₁, R₂, R₄, R₅, R₆, R₇, and R₈ is independently selected from the group consisting of substituted or unsubstituted alkyl, substituted or substituted heteroalkyl, substituted or substituted alkenyl, alkynyl, substituted or unsubstituted cycloalkyl, substituted or unsubstituted cycloheteroalkyl, substituted or unsubstituted aryl, substituted or unsubstituted heteroaryl, arylalkyl, arylalkynyl, alkoxyl, acyloxyl, aryloxyl, arylalkyloxyl, cycloalkylalkyloxyl, cycloalkyloxyl, alkoxyalkyl, alkoxyalkoxyl, aminoalkoxyl, mono- or di-alkylaminoalkoxyl, alkoxycarbonyl, carboxyl, halo, amino, alkylamino, acylamino, arylamino, sulfonyl, arylmercapto, alkylmercapto, hydroxyl, hydroxyalkyl, hydroxycycloalkyl, alkoxycycloalkyl, aminoalkyl, alkylaminoalkyl, cyano, nitro, CF₃, —COR₁₂, —COOR₁₃, and —OR₁₄, wherein R₁₂, R₁₃, and R₁₄ are as defined above;
R₃ is selected from the group consisting of H, substituted or unsubstituted alkyl, substituted or substituted heteroalkyl, substituted or substituted alkenyl, alkynyl, substituted or unsubstituted cycloalkyl, substituted or unsubstituted cycloheteroalkyl, substituted or unsubstituted aryl, substituted or unsubstituted heteroaryl, aralkyl, hydroxyl, —COR₁₂, —COOR₁₃, —OR₁₄, wherein R₁₂, R₁₃, and R₁₄ are as defined above; and
stereoisomers, prodrugs, and pharmaceutically acceptable salts thereof.

2. The method of claim 1, wherein the compound of formula (I) has the following structure: 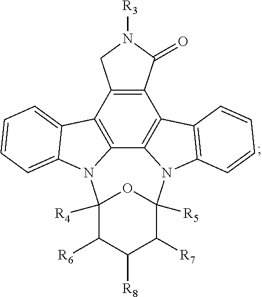
wherein:
R₃ is selected from the group consisting of H, substituted or unsubstituted alkyl, hydroxyl, and alkoxyl; and
R₄, R₅, R₆, R₇, and R₈ are each independently selected from the group consisting of H, substituted or unsubstituted alkyl, alkoxyl, and aminoalkyl.

3. The method of claim 2, wherein the compound of formula (I) has the following structure:

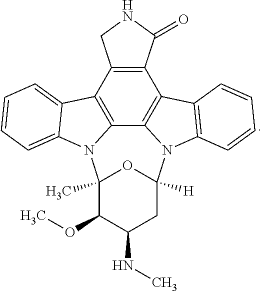 [SMILES: CN[C@@H]1C[C@H]2O[C@@](C)([C@@H]1OC)n1c3ccccc3c3c4c(c5c6ccccc6n2c5c31)C(=O)NC4]

4. The method of claim 1, wherein the compound of formula (II) has the following structure: 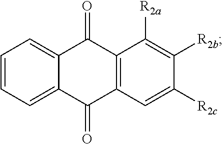
wherein:
R₂ₐ, R_{2b}, and R_{2c} are selected from the group consisting of hydroxyl, alkoxyl, and —COR₁₂, wherein C₁₂ is selected from the group consisting of H, substituted or unsubstituted alkyl.

5. The method of claim 4, wherein the compound of formula (II) has the following structure:

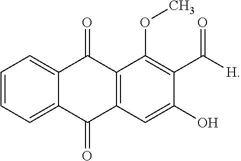

6. The method of claim 1, wherein the compound of formula (III) has the following structure: 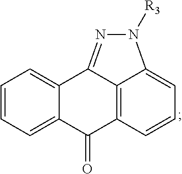
wherein R₃ is selected from the group consisting of H, substituted or unsubstituted alkyl, hydroxyl, and alkoxyl.

7. The method of claim 6, wherein the compound of formula (III) has the following structure:

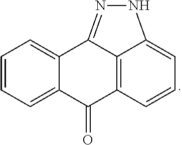 [SMILES: O=C1c2ccccc2-c2n[nH]c3cccc1c23]

8. The method of claim 1, wherein the compound of formula (IV) has the following structure: 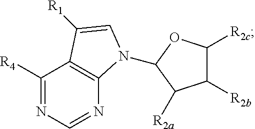
wherein:
R₁ is halo;
R₂ₐ, R_{2b}, and R_{2c} are each independently selected from the group consisting of H, alkyl or unsubstituted alkyl, hydroxyl, alkoxyl, and hydroxyalkyl; and
R₄ is selected from the group consisting of H, alkyl or unsubstituted alkyl, hydroxyl, alkoxyl, and amino.

9. The method of claim 8, wherein the compound of formula (IV) has the following structure:

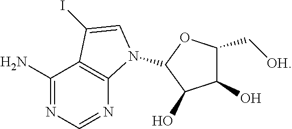

10. The method of claim 1, wherein the compound of formula (V) has the following structure: 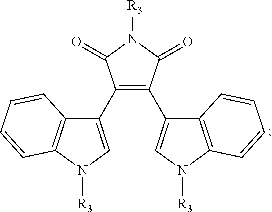
wherein:
each R₃ is independently selected from the group consisting of H, substituted or unsubstituted alkyl, and substituted or substituted heteroalkyl.

11. The method of claim 10, wherein the compound of formula (V) is selected from the group consisting of:

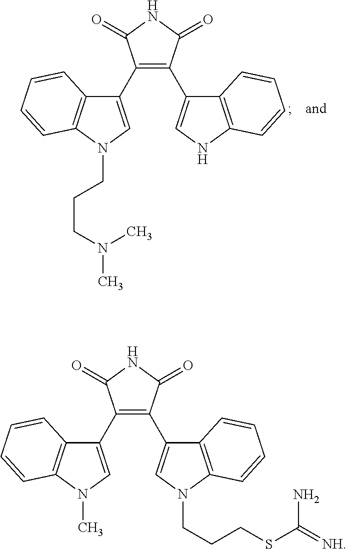 [SMILES: CN(C)CCCn1cc(C2=C(c3c[nH]c4ccccc34)C(=O)NC2=O)c2ccccc21 ; Cn1cc(C2=C(c3cn(CCCSC(=N)N)c4ccccc34)C(=O)NC2=O)c2ccccc21]

12. The method of claim 1, wherein the compound of formula (VI) has the following structure: 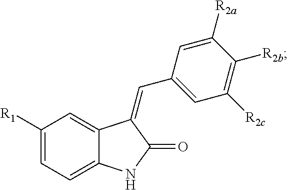
wherein:
R₁ is halo; and
R₂ₐ, R_{2b}, and R_{2c} are each independently selected from the group consisting of H, substituted or unsubstituted alkyl, hydroxyl, alkoxy, and halo.

13. The method of claim 12, wherein the compound of formula (VI) has the following structure:

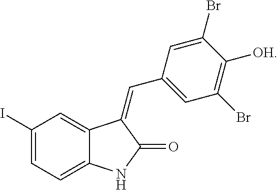 [SMILES: O=C1Nc2ccc(I)cc2/C1=C/c1cc(Br)c(O)c(Br)c1]

14. The method of claim 1, wherein the compound of formula (VII) has the following structure: 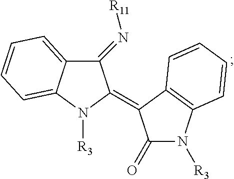
wherein:
each R₃ is independently selected from the group consisting of H, substituted or unsubstituted alkyl, hydroxyl, and alkoxyl; and
R₁₁ is selected from the group consisting of H, substituted or unsubstituted alkyl, hydroxyl, and alkoxyl.

15. The method of claim 14, wherein the compound of formula (VII) has the following structure:

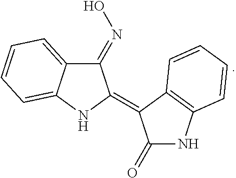 [SMILES: O=C1Nc2ccccc2/C1=C1/Nc2ccccc2/C1=N\O]

16. A method for treating a disorder or a condition that can be treated by inhibiting LRRK2 activity in a subject in need of treatment thereof, the method comprising administering to the subject a therapeutically effective amount of a compound of formulae (I-VII): 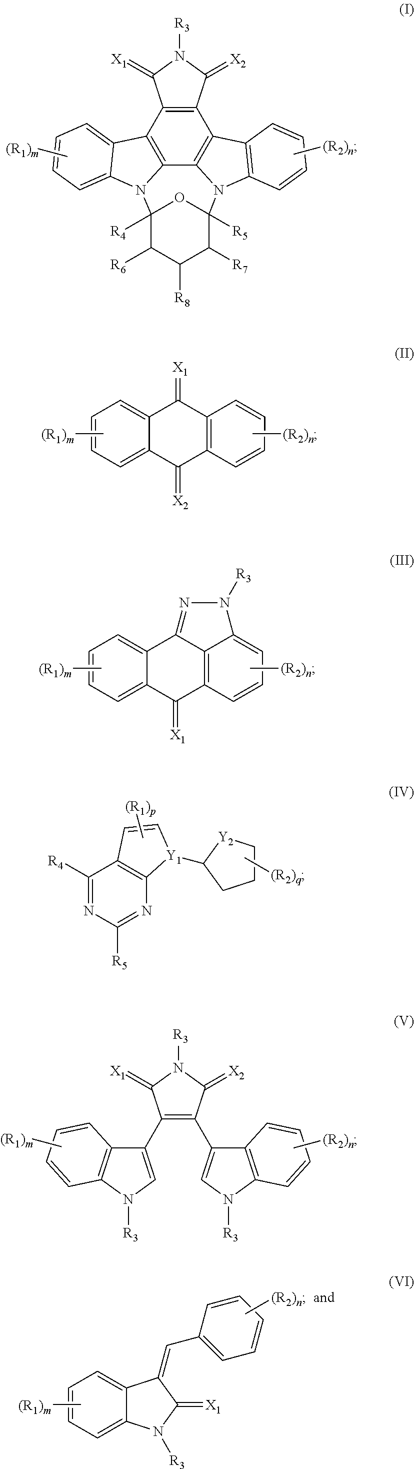 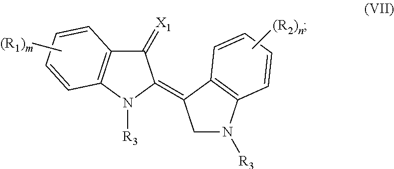
wherein:
p is an integer selected from the group consisting of 0, 1, and 2;
q is an integer selected from the group consisting of 0, 1, 2, and 3;
m and n are each an integer independently selected from the group consisting of 0, 1, 2, 3, and 4;
each (=X₁) and (=X₂) can be present or absent and, when present, each X₁ and X₂ is independently selected from the group consisting of O, S, CR₉R₁₀, and NR₁₁,
wherein R₉, R₁₀, and R₁₁ are each independently selected from the group consisting of H, substituted or unsubstituted alkyl, substituted or substituted heteroalkyl, substituted or substituted alkenyl, alkynyl, substituted or unsubstituted cycloalkyl, substituted or unsubstituted cycloheteroalkyl, substituted or unsubstituted aryl, substituted or unsubstituted heteroaryl, aralkyl, hydroxyl, —COR₁₂, —COOR₁₃, —OR₁₄, wherein R₁₂, R₁₃, and R₁₄ are each independently selected from the group consisting of H, substituted or unsubstituted alkyl, substituted or substituted heteroalkyl, substituted or substituted alkenyl, alkynyl, substituted or unsubstituted cycloalkyl, substituted or unsubstituted cycloheteroalkyl, substituted or unsubstituted aryl, substituted or unsubstituted heteroaryl;
Y₁ is selected from the group consisting of N, and CR₉, wherein R₉ is as defined above;
Y₂ is selected from the group consisting of O, S, CR₉R₁₀, and NR₁₁, wherein R₉, R₁₀, and R₁₁ are as defined above;
each R₁, R₂, R₄, R₅, R₆, R₇, and R₈ is independently selected from the group consisting of substituted or unsubstituted alkyl, substituted or substituted heteroalkyl, substituted or substituted alkenyl, alkynyl, substituted or unsubstituted cycloalkyl, substituted or unsubstituted cycloheteroalkyl, substituted or unsubstituted aryl, substituted or unsubstituted heteroaryl, arylalkyl, arylalkynyl, alkoxyl, acyloxyl, aryloxyl, arylalkyloxyl, cycloalkylalkyloxyl, cycloalkyloxyl, alkoxyalkyl, alkoxyalkoxyl, aminoalkoxyl, mono- or di-alkylaminoalkoxyl, alkoxycarbonyl, carboxyl, halo, amino, alkylamino, acylamino, arylamino, sulfonyl, arylmercapto, alkylmercapto, hydroxyl, hydroxyalkyl, hydroxycycloalkyl, alkoxycycloalkyl, aminoalkyl, alkylaminoalkyl, cyano, nitro, CF₃, —COR₁₂, —COOR₁₃, and —OR₁₄, wherein R₁₂, R₁₃, and R₁₄ are as defined above;
R₃ is selected from the group consisting of H, substituted or unsubstituted alkyl, substituted or substituted heteroalkyl, substituted or substituted alkenyl, alkynyl, substituted or unsubstituted cycloalkyl, substituted or unsubstituted cycloheteroalkyl, substituted or unsubstituted aryl, substituted or unsubstituted heteroaryl, aralkyl, hydroxyl, —COR₁₂, —COOR₁₃, —OR₁₄, wherein R₁₂, R₁₃, and R₁₄ are as defined above; and
stereoisomers, prodrugs, and pharmaceutically acceptable salts thereof.

17. The method of claim 16, wherein the compound of formula (I) has the following structure: 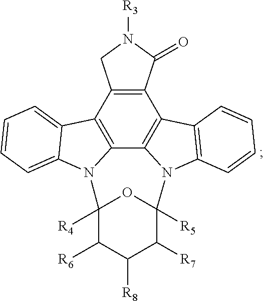
wherein:
R₃ is selected from the group consisting of H, substituted or unsubstituted alkyl, hydroxyl, and alkoxyl; and
R₄, R₅, R₆, R₇, and R₈ are each independently selected from the group consisting of H, substituted or unsubstituted alkyl, alkoxyl, and aminoalkyl.

18. The method of claim 17, wherein the compound of formula (I) has the following structure:

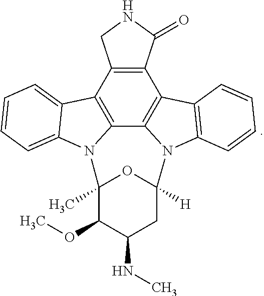 [SMILES: CN[C@@H]1C[C@H]2O[C@@](C)([C@@H]1OC)n1c3ccccc3c3c4c(c5c6ccccc6n2c5c31)C(=O)NC4]

19. The method of claim 16, wherein the compound of formula (II) has the following structure: 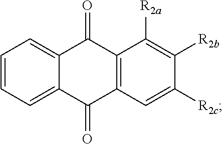
wherein:
R₂ₐ, R_{2b}, and R_{2c} are selected from the group consisting of hydroxyl, alkoxyl, and —COR₁₂, wherein C₁₂ is selected from the group consisting of H, substituted or unsubstituted alkyl.

20. The method of claim 19, wherein the compound of formula (II) has the following structure:

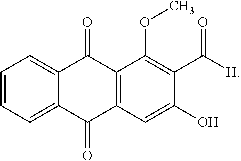

21. The method of claim 16, wherein the compound of formula (III) has the following structure: 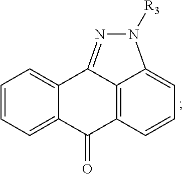
wherein R₃ is selected from the group consisting of H, substituted or unsubstituted alkyl, hydroxyl, and alkoxyl.

22. The method of claim 21, wherein the compound of formula (III) has the following structure:

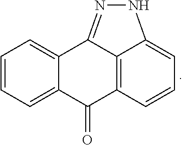 [SMILES: O=C1c2ccccc2-c2n[nH]c3cccc1c23]

23. The method of claim 16, wherein the compound of formula (IV) has the following structure: 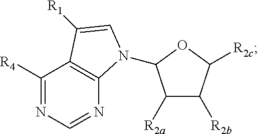
wherein:
R₁ is halo;
R₂ₐ, R_{2b}, and R_{2c} are each independently selected from the group consisting of H, alkyl or unsubstituted alkyl, hydroxyl, alkoxyl, and hydroxyalkyl; and
R₄ is selected from the group consisting of H, alkyl or unsubstituted alkyl, hydroxyl, alkoxyl, and amino.

24. The method of claim 23, wherein the compound of formula (IV) has the following structure:

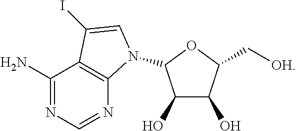

25. The method of claim 16, wherein the compound of formula (V) has the following structure: 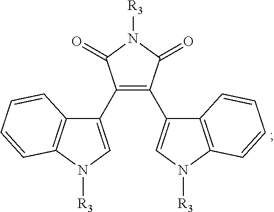
wherein:
each R₃ is independently selected from the group consisting of H, substituted or unsubstituted alkyl, and substituted or substituted heteroalkyl.

26. The method of claim 25, wherein the compound of formula (V) is selected from the group consisting of:

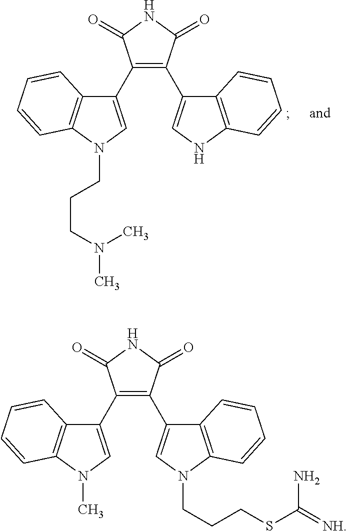 [SMILES: CN(C)CCCn1cc(C2=C(c3c[nH]c4ccccc34)C(=O)NC2=O)c2ccccc21 ; Cn1cc(C2=C(c3cn(CCCSC(=N)N)c4ccccc34)C(=O)NC2=O)c2ccccc21]

27. The method of claim 16, wherein the compound of formula (VI) has the following structure: 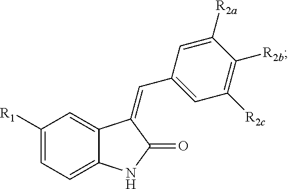
wherein:
R₁ is halo; and
R₂ₐ, R_{2b}, and R_{2c} are each independently selected from the group consisting of H, substituted or unsubstituted alkyl, hydroxyl, alkoxy, and halo.

28. The method of claim 27, wherein the compound of formula (VI) has the following structure:

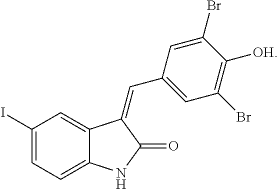 [SMILES: O=C1Nc2ccc(I)cc2/C1=C/c1cc(Br)c(O)c(Br)c1]

29. The method of claim 16, wherein the compound of formula (VII) has the following structure: 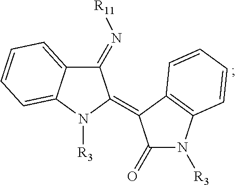
wherein:
each R₃ is independently selected from the group consisting of H, substituted or unsubstituted alkyl, hydroxyl, and alkoxyl; and
R₁₁ is selected from the group consisting of H, substituted or unsubstituted alkyl, hydroxyl, and alkoxyl.

30. The method of claim 29, wherein the compound of formula (VII) has the following structure:

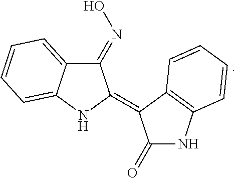 [SMILES: O=C1Nc2ccccc2/C1=C1/Nc2ccccc2/C1=N\O]

31. The method of claim 16, wherein the disorder or condition that can be treated by inhibiting LRRK2 activity comprises a neurodegenerative disease.

32. The method of claim 31, wherein the neurodegenerative disease is selected from the group consisting of Alexander's disease, Alper's disease, Alzheimer's disease, amyotrophic lateral sclerosis, ataxia telangiectasia, Batten disease, bovine spongiform encephalopathy, Canavan disease, Cockayne syndrome, corticobasal degeneration, Creutzfeldt-Jakob disease, Huntington's disease, HIV-associated dementia, Kennedy's disease, Krabbe's disease, lewy body dementia, Machado-Joseph disease, multiple sclerosis, multiple system atrophy, narcolepsy, neuroborreliosis, Parkinson's disease, Pelizaeus-Merzbacher Disease, Pick's disease, primary lateral sclerosis, prion diseases, Refsum's disease, Sandhoff's disease, Schilder's disease, subacute combined degeneration of spinal cord secondary to pernicious anaemia, schizophrenia, spinocerebellar ataxia, spinal muscular atrophy, Steele-Richardson-Olszewski disease, and tabes dorsalis.

33. The method of claim 32, wherein the neurodegenerative disease is Parkinson's disease.

34. The method of claim 16, wherein the disorder or condition that can be treated by inhibiting LRRK2 activity comprises an autoimmune disease.

35. The method of claim 34, wherein autoimmune disease comprises Crohn's disease.

36. The method of claim 16, wherein the treating of the disorder or a condition that can be treated by inhibiting LRRK2 activity comprises preventing the disorder or condition, slowing the onset or progression of the disease or condition, alleviating one or more symptoms of the disease or condition, or any combination thereof.
